(19) United States
(12) Patent Application Publication
(10) Pub. No.: US 2016/0158154 A1
(43) Pub. Date: Jun. 9, 2016

(54) PROTEIN VESICLES AND METHODS OF MAKING AND USING THEREOF

(71) Applicant: Georgia Tech Research Corporation, Atlanta, GA (US)

(72) Inventors: Julie A. Champion, Atlanta, GA (US); Won Min Park, Atlanta, GA (US)

(21) Appl. No.: 14/961,616

(22) Filed: Dec. 7, 2015

Related U.S. Application Data
(60) Provisional application No. 62/088,050, filed on Dec. 5, 2014.

(51) Int. Cl. A61K 9/127; A61K 49/00

(52) U.S. Cl. CPC A61K 9/1271; A61K 9/1277; A61K 49/0002

(57) ABSTRACT

Hollow, protein vesicles are provided. The protein vesicles can be loaded with desired cargo to be delivered to a subject, for example a human. One embodiment provides a protein vesicle made of a protein membrane surrounding a hollow core. The protein membrane includes a first and a second modular protein amphiphile. The first modular protein amphiphile includes a hydrophobic block and a first hydrophilic protein binding block. The second modular protein amphiphile includes a variable polypeptide block and a second hydrophilic protein binding block that binds to the first hydrophilic protein binding block. The first and second protein amphiphiles self-assemble to form the hollow, protein vesicle.

### CROSS-REFERENCE TO RELATED APPLICATION

This application claims benefit of and priority to U.S. Provisional Application No. 62/088,050 filed Dec. 5, 2014, which is hereby incorporated herein by reference in its entirety.

### STATEMENT REGARDING FEDERALLY SPONSORED RESEARCH OR DEVELOPMENT

This invention was made with government support under grant number 1032413, awarded by the National Science Foundation. The government has certain rights in the invention.

### REFERENCE TO SEQUENCE LISTING

The Sequence Listing submitted Dec. 7, 2015 as a text file named “GTRC_6823_ST25” created on Dec. 7, 2015, and having a size of 20,810 bytes is hereby incorporated by reference pursuant to 37 C.F.R. §1.52(e)(5).

### FIELD OF THE INVENTION

The invention is generally directed to vesicles made from proteins and methods of making and using them.

### BACKGROUND OF THE INVENTION

Effectively treating an illness or disease can be significantly dependent on the ability of delivering a medicine or compound to an affected tissue or site of a pathology. Systemic administration of therapeutic agents typically requires administering an artificially high dose of an agent to ensure that an effective amount of the agent actually reaches the affected tissue or site. Administering such artificially high doses of agents can contribute to unwanted side-effects which in some cases can cause more harm than good. To minimize drug degradation and loss, to prevent harmful side-effects and to increase drug bioavailability by enhancing drug targeting specificity, various drug delivery and drug targeting systems are currently under development. (Rani, D., IRJP, 4(1):6-12 (2013)).

Self-assembled vesicles such as liposomes are of great interest in drug delivery and imaging, given their ability to sequester large payloads of hydrophilic or hydrophobic agents (Vargo K B, et al., Proc. Natl. Acad. Sci. U.S.A., 109, 11657 (2012)). However, problems with liposomal drug delivery systems include their rapid clearance from circulation due to uptake by the reticuloendothelial system; leakage of encapsulated drugs; batch to batch variation; and physical and chemical instability.

Recombinant proteins have also been developed to self-assemble into vesicles (Vargo K B, et al., Proc. Natl. Acad. Sci, U.S.A., 109, 11657 (2012)). They are biocompatible and biodegradable and can offer biofunctionality through incorporation of peptide sequences or folded proteins. Self-assembly of folded proteins, in many examples, provides a versatile method to fabricate functional biomaterials for a range of applications (Hudalla G A, et al., Nat. Mater., 13, 829 (2014); Leng Y, et al., Angew. Chem., Int. Ed., 49, 7243 (2010)). However, direct incorporation of folded, functional and biologically relevant moieties into protein amphiphiles can prevent conformational arrangement of chains during vesicle formation, and their molecular weight might be limited. Furthermore, organic solvents, which are typically added to dissolve amphiphilic proteins or polypeptides (Bellomo E G, et al., Nat. Mater., 3, 244 (2004); Vargo K B, et al., Proc. Natl. Acad. Sci. USA., 109, 11657 (2012)), can hamper biological activity of incorporated folded proteins. For these reasons, vesicles of folded recombinant proteins are underdeveloped (Vargo K B, et al., Proc. Natl. Acad. Sci. USA., 109, 11657 (2012)). In fact, folded, globular proteins have been incorporated into vesicles only as hybrid forms of protein synthetic polymers (Amado E, et al., ACS Macro Lett., 1, 1016 (2012); Liu Z, ACS Appl. Mater. Interfaces, 6(4): 2393-2400 (2014)).

Therefore, it is an object of the invention to provide engineered proteins that can assemble into vesicles.

It is another object of the invention to provide compositions and methods for targeted delivery of cargo in protein vesicles.

### SUMMARY OF THE INVENTION

Hollow, protein vesicles are provided. The protein vesicles can be loaded with desired cargo to be delivered to a subject, for example a human. One embodiment provides a protein vesicle made of a protein membrane surrounding a hollow core. The protein membrane includes a first and a second modular protein amphiphile. The first modular protein amphiphile includes a hydrophobic block and a first hydrophilic protein binding block. The second modular protein amphiphile includes a variable polypeptide block and a second hydrophilic protein binding block that binds to the first hydrophilic protein binding block. The first and second protein amphiphiles self-assemble to form the hollow, protein vesicle.

In one embodiment, the hydrophobic block of the first modular protein amphiphile includes an elastin-like polypeptide, and the first and second hydrophilic protein binding blocks include a leucine zipper motif. Preferred leucine zipper motifs include arginine-rich and glutamic acid-rich leucine zipper motifs. The second hydrophilic block can include a catalytic domain of an enzyme, a targeting moiety, a fluorescent polypeptide, a Human Leukocyte Antigen polypeptide, a T cell receptor polypeptide, a detectable label, a globular polypeptide, an immunostimulatory polypeptide, or combinations thereof.

In certain embodiments, the enzyme is selected from the group consisting of a dehydrogenase, a reductase, a protease, a synthetase.

In other embodiments, the targeting moiety is selected from the group consisting of an antibody, aptamer, ligand receptor, and fusion protein. The antibody is selected from the group consisting of a monoclonal antibody, a polyclonal antibody, a diabody, a single chain antibody, and antigen binding fragments thereof.

Another embodiment provides a method for making hollow, protein vesicles by combining a plurality of first and second modular protein amphiphiles in a solution on ice and adjusting the salt concentration to promote or allow inverse phase transition of the protein amphiphiles. The salt-adjusted solution is then incubated at room temperature to form hollow, protein vesicles.

Still another embodiment provides a loaded, protein vesicle having a protein membrane surrounding a core, wherein the protein membrane includes a first and a second modular protein amphiphile, wherein the first modular protein amphiphile comprises a hydrophobic block and a first hydrophilic protein binding block and the second modular protein amphiphile include a variable polypeptide block and a second hydrophilic protein binding block that binds to the first hydrophilic protein binding block. The first and second protein amphiphiles self-assemble around cargo to form the protein vesicle with the cargo loaded in the core of the protein vesicle. The cargo can include a drug, a protein, a contrast agent, nanoparticles, a fluorophore, a radiolabel, a therapeutic compound, an antioxidant, a growth factor, a cytokine, a chemoattractant, a nucleic acid or a combination thereof. The drug can be selected from the group consisting of a cytocidal compound, a chemotherapy agent, a cellular metabolism blocker, a cell cycle inhibitor, or a combination thereof. The nucleic acid can encode a functional protein. In other embodiments, the nucleic acid is selected from the group consisting of double stranded DNA, RNA, siRNA, microRNA, single stranded DNA, and anti-sense DNA.

### BRIEF DESCRIPTION OF THE DRAWINGS

FIG. 1A is a drawing of recombinant diblock copolypeptide, Z_{R}-ELP. FIG. 1B is a drawing of recombinant diblock copolypeptide, mCherry-Z_{E}. FIG. 1C is a drawing of recombinant diblock copolypeptide, EGFP-Z_{E}. FIG. 1D is an image of SDS-PAGE gel from Coomassie Blue staining. Ladder protein markers are on the left-most lane, and Z_{R}-ELP, mCherry-Z_{E}, and EGFP-Z_{E} samples are on lane labeled as 1, 2, and 3, respectively. FIG. 1E is a fluorescence image of the SDS-PAGE gel at filter settings of excitation 532 nm/emission 610 nm, showing the fluorescence of mCherry-Z_{E}. FIG. 1F is a fluorescence image of the SDS-PAGE gel at filter settings of excitation 488 nm/emission 526 nm, showing the fluorescence of EGFP-Z_{E}. FIG. 1G is a diagram of a representative method for producing hollow protein vesicles.

FIG. 2A is a drawing of the “rod-coil” structure of Z_{R}-ELP homodimer and the “globule-rod-coil” structures of mCherry-Z_{E}/Z_{R}-ELP and EGFP-Z_{E}/Z_{R}-ELP) protein complexes prepared in solution below the inverse phase transition temperature of ELP. FIG. 2B is a dot plot showing molar ellipticity (θ, 10⁻³ deg cm² dmol⁻¹) over wavelength (nm) in a circular dichroism spectrum of protein complexes Z_{R}-ELP (in black dots), mCherry-Z_{E}/Z_{R}-ELP (in red dots), and EGFP-Z_{E}/Z_{R}-ELP (in green dots) the inverse phase transition temperature of ELP.

FIG. 3 is a series of photographs showing the turbidity of protein solution placed from 4° C. to 25° C. over time (minutes).

FIG. 4A is a confocal micrograph of mCherry-Z_{E}/Z_{R}-ELP vesicles formed from 1.5 μM of mCherry-Z_{E} and 30 μM Z_{R}-ELP in a 0.30 M salt solution, and an inset of a close-up image. Scale bars are 10 and 1 μm (inset), respectively. FIG. 4B is a confocal micrograph of EGFP-Z_{E}/Z_{R}-ELP vesicles formed from 0.6 μM of EGFP-Z_{E} and 30 μM Z_{R}-ELP in a 0.91 M salt solution, and an inset of a close-up image. Scale bars are 10 and 1 μm (inset), respectively. FIG. 4C is a confocal micrograph of vesicles formed from 0.3 mCherry-Z_{E}, 0.3 μM EGFP-Z_{E} and 30 μM Z_{R}-ELP in a 0.91 M salt solution, and an inset of a close-up image. Scale bars are 10 and 1 μm (inset), respectively.

FIG. 5 is a line graph showing the fluorescence intensity (a.u.) over distance (μm) of a mCherry-Z_{E}/Z_{R}-ELP vesicle formed at 0, 5, 15, to 30 minutes after placed at room temperature.

FIG. 6A is a scanning electron microscopy (SEM) image of the surface of a mCherry-Z_{E}/Z_{R}-ELP vesicle. Scale bar is 1 μm. FIG. 6B is a SEM image of the cross-section of a fractured mCherry-Z_{E}/Z_{R}-ELP vesicle. Scale bar is 1 μm. FIG. 6C is a SEM image of a part of the cross-section of a fractured mCherry-Z_{E}/Z_{R}-ELP vesicle. Scale bar is 100 nm.

FIG. 7A is a confocal micrograph of mCherry-Z_{E}/Z_{R}-ELP coacervate formed from 1.5 μM of mCherry-Z_{E} and 30 μM Z_{R}-ELP in a 0.15 M salt solution. FIG. 7B a confocal micrograph of EGFP-Z_{E}/Z_{R}-ELP coacervate formed from 0.6 μM of mCherry-Z_{E} and 30 μM Z_{R}-ELP in a 0.15 M salt solution.

FIG. 8 is a dot plot showing the turbidity (i.e., optical density at 400 nm) of protein solutions over time (minutes) during inverse phase transition at 25° C. Legends indicate protein solutions of a molar ratio of mCherry-Z_{E} to Z_{R}-ELP (χ), (χ=0.05 or 0), at different salt concentrations (0.15 M or 0.30M).

FIG. 9 is a drawing of the molecular packing (packing parameter, P) of the truncated cone models for Z_{R}-ELP homodimer and mCherry-Z_{E}/Z_{R}-ELP heterodimer.

FIG. 10A is a line plot showing the number-based distribution of the hydrodynamic diameter (d_{H}, μm) of vesicles with different molar ratios (χ) of mCherry-Z_{E} to Z_{R}-ELP (top), 0.01, 0.02, 0.03, 0.05, 0.1; from right to left; and EGFP-Z_{E} to Z_{R}-ELP (bottom), 0.01, 0.02, 0.03, 0.05 from left to right. FIG. 10B is a line plot showing the hydrodynamic diameter (d_{H}, μm) over the molar ratio (χ) of mCherry-Z_{E} to Z_{R}-ELP and that of EGFP-Z_{E} to Z_{R}-ELP, corresponding to the result in FIG. 10A.

FIG. 11A is a confocal micrograph of mCherry-Z_{E}/Z_{R}-ELP vesicles encapsulating the coacervate phase of EGFP-Z_{E} and Z_{R}-ELP. The structure is a self-assembly of the mixture of 1.5 μM of mCherry-Z_{E}, 0.6 μM of mCherry-Z_{E}, and 30 μM Z_{R}-ELP in a 0.45 M salt solution. Scale bar is 1 μm. FIG. 11B is a fluorescence intensity profile of the self-assembled structure corresponding to FIG. 11A. FIG. 11C is a dot plot showing the turbidity (i.e., optical density at 400 nm) of protein solutions over time (minutes) during inverse phase transition at 25° C. Legends indicate protein solutions containing 1.5 μM mCherry-Z_{E} (red dots), 0.6 μM EGFP-Z_{E} (green dots), and both (blue dots) at a salt concentration of 0.45 M with 30 μM Z_{R}-ELP.

FIG. 12 is a drawing of the proposed model of single-layer vesicular membrane.

FIG. 13 is a confocal micrograph of mCherry-Z_{E}/Z_{R}-ELP vesicles encapsulating a small molecule, fluorescein. Scale bars are 10 and 1 μm (inset), respectively.

FIG. 14 is a confocal micrograph of mCherry-Z_{E}/Z_{R}-ELP vesicles encapsulating fluorescent polystyrene nanoparticles with a diameter of 500 nm. Scale bars are 10 and 1 μm (inset), respectively.

### DETAILED DESCRIPTION OF THE INVENTION

### I. Definitions

As used herein, the term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express nucleic acids or polypeptides that are not found within the native (non-recombinant) form of the cell or express native genes that are otherwise abnormally expressed, under expressed, over expressed or not expressed at all. These polypeptides or proteins expressed are also called fusion polypeptides or fusion proteins.

As used herein, the terms “protein” and “polypeptide” are used interchangeably herein. The conventional one-letter or three-letter code for amino acid residues is used herein. The peptides can be all L-stereo configuration, all D-stereo configuration, or a mixture of L- and D-stereo configuration.

As used herein, the term “vesicle” means a hollow particle which may be nano or micro sized. Vesicles carry components encapsulated in the interior, entrapped in the membrane or presented on the surface of the membrane facing outward. Vesicles are formed by an appropriate choice of amphiphilic proteins and/or polypeptides that form the membrane. Some vesicles are formed with single-layer membrane, while others are formed with double-layer membrane.

As used herein, the term “hydrophilic” refers to the property of having affinity for water. For example, hydrophilic polymers (e.g. hydrophilic protein) are polymers which are primarily soluble in aqueous solutions and/or have a tendency to absorb water. In general, the more hydrophilic a polymer is, the more that polymer tends to dissolve in, mix with, or be wetted by water.

As used herein, the term. “hydrophobic” refers to the property of lacking affinity for, or even repelling water. For example, the more hydrophobic a polymer (e.g. hydrophobic protein), the more that polymer tends to not dissolve in, not mix with, or not be wetted by water.

As used herein, the term “amphiphile” refers to a molecule combining hydrophilic and lipophilic (hydrophobic) properties.

As used herein, the term “self-assembly” refers to the spontaneous formation of a composition or a structure in a medium containing the appropriate components.

As used herein, “phase transition” and “inverse phase transition” refer to the change of solubility property depending on the ambient temperature, pressure, salt concentration, or a combination thereof. Substances (e.g. elastin-like peptides, ELP) capable of undergoing phase transition show a lower critical solution temperature (or LCST), which above the so-called phase transition temperature (Tₜ) results in the transition from a soluble to an insoluble form in a narrow range of temperatures (˜2° C.) in a reversible process known as coacervation. The phase transition temperature is maximum value defined as the temperature in which ELP aggregation occurs, causing solution turbidity to increase to half its initial value, accompanied by an increase in the temperature of the solution. In solutions with a temperature lower than Tₜ, free protein or polypeptide chains remain in an unordered state showing full hydration (the soluble form). In solutions with temperatures exceeding Tₜ, the situation is different: polymer chains show a more ordered structure (known as the b-spiral), stabilized by hydrophobic interactions and intramolecular type b structures increasing the association of polymer chains.

As used herein, the term “coacervate” refers to a liquid/liquid phase separation occurring in protein mixtures, resulting in the formation of two liquid phases: a polyelectrolyte-rich, so called coacervate phase, and a dilute continuous phase mostly devoid of polyelectrolyte. There are two types of coacervation, namely simple coacervation and complex coacervation, depending on whether the coacervate phase is constituted of a single polymer or of an ionic complex of two oppositely charged polymers.

As used herein, the term “transition temperature” or “Tₜ” refers to the temperature above which a polymer (for example, an ELP) that undergoes an inverse temperature transition is insoluble in an aqueous system (e.g., water, physiological saline solution, blood, or serum), and below which such a polymer is soluble in the aqueous system.

As used herein, the terms “elastin-like peptide”, “elastin-like polypeptide”, and “elastin-like protein” are used interchangeably and refer to polypeptides comprising polymers of the pentapeptide Val-Pro-Gly-Xaa-Gly (SEQ ID NO: 1), where the “guest residue” Xaa is any amino acid except Pro.

As used herein, the term “polypeptide” refers to a chain of amino acids of any length, regardless of modification (e.g., phosphorylation or glycosylation). The sequences are written left to right in the direction from the amino (N) to the carboxyl (C) terminus. In accordance with standard nomenclature, amino acid residue sequences axe denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gin, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, a “variant” polypeptide contains at least one amino acid sequence alteration as compared to the amino acid sequence of the corresponding wild-type polypeptide.

As used herein, an “amino acid sequence alteration” can be, for example, a substitution, a deletion, or an insertion of one or more amino acids.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences

As used herein, an “expression control sequence” is a DNA sequence that controls and regulates the transcription and/or translation of another DNA sequence.

As used herein, “operably linked” means incorporated into a gentic construct so that expression control sequences effectively control expression of a coding sequence of interest.

As used herein, a “fragment” of a polypeptide refers to any subset of the polypeptide that is a shorter polypeptide of the full length protein. Generally, fragments will be five or more amino acids in length.

As used herein, “valency” refers to the number of binding sites available per molecule.

As used herein, the term “host cell” refers to prokaryotic and eukaryotic cells into which a recombinant expression vector can be introduced.

As used herein, “transformed” and “transfected” encompass the introduction of a nucleic acid (e.g., a vector) into a cell by a number of techniques known in the art.

As used herein, the term “gene” or “genes” refers to isolated or modified nucleic acid sequences, including both RNA and DNA, that encode genetic information for the synthesis of a whole RNA, a whole protein, or any portion of such whole RNA or whole protein. Genes that are not naturally part of a particular organism's genome are referred to as “foreign genes”, “heterologous genes” or “exogenous genes” and genes that are naturally a part of a particular organism's genome are referred to as “endogenous genes”. The term “gene” as used with reference to genomic DNA includes intervening, non-coding regions as well as regulatory regions and can include 5′ and 3′ ends.

As used herein, the term “antibody” is meant to include both intact molecules as well as fragments thereof that include the antigen-binding site. These include Fab and F(ab′)₂ fragments which lack the Fe fragment of an intact antibody.

As used herein, the terms “incorporate” and “encapsulate” refer to incorporating, formulating, or otherwise including an active agent into and/or onto a composition that allows for inclusion and/or release, such as sustained release, of such agent in the desired application. The terms contemplate any manner by which a therapeutic agent or other material is incorporated, including, for example: attached (by covalent, ionic, or other binding interaction), physical admixture, enveloping the agent in a coating layer, incorporated, distributed throughout the protein structure, appended to the surface, encapsulated inside the protein vesicle, etc. The term “co-incorporation” or “co-encapsulation” refers to the incorporation of a therapeutic agent or other material and at least one other therapeutic agent or other material in a subject composition.

As used herein, the term “active agent” refers to a physiologically or pharmacologically active substance that acts locally and/or systemically in the body and/or outside a body in an applicable environment. An active agent is a substance that treats (e.g., therapeutic agent), prevents (e.g., prophylactic agent), or diagnoses (e.g., diagnostic agent) of a disease or disorder, or a substance that catalyzes (e.g., enzymes).

As used herein, the term “excipient” refers to an organic or inorganic ingredient, natural or synthetic inactive ingredient M a formulation, with which one or more active ingredients are combined.

As used herein, the term “pharmaceutically acceptable” means a non-toxic material that does not interfere with the effectiveness of the biological activity of the active ingredients.

The terms “individual”, “host”, “subject”, and “patient” are used interchangeably herein, and refer to a mammal, including, but not limited to, humans, primates, rodents, such as mice and rats, and other laboratory animals.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

As used herein the term “nanoparticle” generally refers to a particle of any shape having a diameter from about 1 nm up to, but not including, about 1 micron, more preferably from about 5 nm to about 500 nm, most preferably from about 5 nm to about 100 nm. Nanoparticles having a spherical shape are generally referred to as “nanospheres”.

As used herein the term “administering” or “exposing” is the delivery of a vesicle and optionally the release of a therapeutic or other cargo molecule to a subject or an environment. The therapeutic for a subject is administered by a route determined to be appropriate for a particular subject by one skilled in the art. For example, the therapeutic is administered orally, parenterally (for example, intravenously), by intramuscular injection, by intraperitoneal injection, intratumorally, by inhalation, or transdermally. The exact amount of therapeutic required will vary from subject to subject, depending on the age, weight and general condition of the subject, the severity of the neurological condition that is being treated, the particular therapeutic used, its mode of administration, and the like. An appropriate amount may be determined by one of ordinary skill in the art using only routine experimentation given the teachings herein or by knowledge in the art without undue experimentation.

As used herein, “treatment” or “treating” means to administer a composition to a subject or a system with an undesired condition (e.g., cancer or other proliferative disorder). The condition can include a disease.

The use of the terms “a”, “an”, “the”, and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx.+/−10%; in other embodiments the values may range in value either above or below the stated value in a range of approx.+/−5%; in other embodiments the values may range in value either above or below the stated value in a range of approx.+/−2%; in other embodiments the values may range in value either above or below the stated value in a range of approx.+/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II. Protein Vesicles

Protein vesicles are formed with protein or polypeptide amphiphiles or fusion proteins thereof. One embodiment provides protein vesicles that are formed by the self-assembly of at least two protein amphiphiles. Another embodiment provides protein vesicles that are formed by the self-assembly of three or more protein amphiphiles. The protein vesicles are typically made of a protein membrane that encloses a hollow core. The protein is typically a single layer membrane. The hollow core can be loaded with cargo, for example as the protein amphiphiles self-assemble into the protein vesicles. Representative cargo includes, but is not limited a drug, a protein, a contrast agent, nanoparticles, a fluorophore, a radiolabel, a therapeutic compound, an antioxidant, a growth factor, a cytokine, a chemoattractant, a nucleic acid or a combination thereof. Because the protein vesicles can be loaded with cargo, the protein vesicles can be used as delivery vehicles to target the delivery of the cargo to a desired cell, tissue, organ, a tumor, a wound, a site of pathology in a subject.

The protein amphiphiles that form the protein vesicles are preferably modular proteins that contain at least two domains. The protein vesicles can be formed from homodimers or heterodimers. A first protein amphiphile includes a hydrophobic block and a protein binding block. A preferred hydrophobic block for the first protein amphiphile includes an elastin-like polypeptide (ELP) motif. A preferred protein binding block for the first protein amphiphile includes a leucine zipper motif, for example an arginine-rich zipper motif.

The second protein amphiphile includes a protein binding block that binds to or hybridizes with the protein binding block of the first protein amphiphile. An exemplary protein binding block of the second protein amphiphile includes a leucine zipper motif, for example a glutamic acid-rich leucine zipper motif. The second block of the second protein amphiphile includes a variable hydrophilic block. This variable hydrophilic block can be a polypeptide, for example a globular polypeptide. The polypeptide can also be an enzyme or catalytic domain of an enzyme, a binding moiety such as an antibody, an aptamer, or combinations thereof.

Protein amphiphiles in addition to the first and second amphiphiles can also be used to form the protein vesicles. These additional protein amphiphiles include a protein binding block such as a leucine zipper and a variable hydrophilic block that can be a polypeptide as discussed above.

One embodiment provides protein amphiphiles that are fusion proteins wherein each block of the fusion protein is from a different source, i.e., a different protein.

The protein amphiphiles, protein moieties, and/or peptide motifs that can be recombinantly engineered or physically/chemically bonded include (i) a functional, full-length protein or a fragment thereof, fused directly, via a linker peptide sequence, or bonded with a di-/multimerization polypeptide domain, (ii) a hydrophobic protein moiety or a hydrophobic peptide motif, or one that can undergo phase transition to become insoluble in the medium, fused directly, via a linker peptide sequence, or bonded with a di-/multimerization polypeptide domain, (iii) a functional protein or its moiety fused directly, via a linker peptide sequence, or bonded with a hydrophobic protein or peptide moiety, or with one that can undergo phase transition to become insoluble in a medium, and (iv) a functional protein or its moiety fused directly, via a linker peptide sequence, or bonded with a di-/multimerization polypeptide domain, further fused or bonded with a hydrophobic protein or peptide moiety, or further with one that can undergo phase transition to become insoluble in a medium.

It is believed that proteins fused directly (or via a linker) to hydrophobic proteins (like ELP) only form micelles, not vesicles. Thus, in one embodiment, the zippers provide enough structural support to create a vesicle, which is larger and has a larger hollow inside space than a micelle.

Vesicles can be formed by a mixture of one or more elements of (i) and (ii), (i) and (iii), (i) and (iv), (ii) and (iii), (ii) and (iv), (iii) alone, (iv) alone, or combinations thereof.

In the embodiment of fusion proteins, a first polypeptide can be placed in the N-terminus or the C-terminus of a second polypeptide. In a further embodiment, branched polypeptides are made by adding an oligo-lysine to the C-terminus of a polypeptide, where the alpha- and epsilon-amino groups of the N-terminal lysine act as the branch points for bonding with two or more hydrophobic or insoluble peptide tails or di-/multimerizable peptide moieties. Some amino acids or all amino acids of a peptide or protein can be L-stereo configuration or D-stereo configuration.

In the embodiment of chemically associated protein or polypeptide moieties, a first polypeptide can be chemically conjugated with a second polypeptide via known linkers. Such linking agents are categorized and can be varied in the chemical reactivity, spacer length, and materials. In one embodiment, carbodiimide-based compounds (e.g., 1-ethyl-3-(3-dimethylaminopropyl)carbodiimide) directly crosslink a carboxyl group of an amino acid of a first polypeptide to an amine group of another amino acid. In another embodiment, maleimide, haloacetyl, pyridyldisulfide, thiosulfonate, or vinylsulfone-based compounds can crosslink sulfhydryl groups (e.g. cysteines). In another embodiment, the linking agents have hetero-bifunctional reactive groups and optionally a spacer arm, e.g. 4-succinimidyloxycarbonyl-α-methyl-α(2-pyridyldithiol)toluene, m-maleimidobenzoyl-N-hydroxysulfosuccinimide ester, sulfosuccinimidyl 4-(N-maleimidophenyl)butyrate, and their variants containing poly(ethylene)glycol as the spacer arm.

There are several advantages to using polypeptides as opposed to synthetic polymers. First, polypeptides made of natural amino acids are likely to maintain biocompatibility throughout the degradation process, breaking down into metabolites excreted through normal metabolic pathways. Second, genetically encoded peptides exhibit molecular weight and sequence uniformity, which are properties that will control pharmacokinetics, transport, biodistribution, and degradation (Ghandehari H, et al., Pharm Res., 15(6):813-815 (1998); Nagarsekar A, et al., J Drug Target., 7(1):11-32 (1999)). Third, targeting moieties such as short peptide segments can be incorporated at the genetic level at predetermined locations on the protein.

A. Proteins or Polypeptides Serving as Hydrophilic Headgroup

The protein vesicles can include a folded, active protein or polypeptide as a hydrophilic headgroup on the membrane. The presence of the biologically active protein can provide functional activities to the protein vesicles.

1. Targeting Signal or Domain

In some embodiments, the protein vesicles include one or more targeting signals or domains. The targeting signal can include a sequence of monomers that facilitates in vivo localization of the molecule. The targeting signal or sequence can be specific for a host, tissue, organ, cell, organelle, an organelle such as the nucleus, or cellular compartment.

In some embodiments, the targeting signal binds to a ligand or receptor which is located on the surface of a target cell such as to bring the protein vesicles and cell membranes sufficiently close to each other to allow penetration of the protein vesicles into the cell or deliver encapsulated agents.

In other embodiments, the vesicle is not endocytosed but the act of binding receptors (especially in a multivalent fashion form multiple ligands on the vesicle surface) can be therapeutic itself, or binding gest the vesicle in contact with the cell so when the small molecule drug leaks out it can go right through the cell membrane even though the vesicle does not go in the cell.

In a preferred embodiment, the targeting molecule is selected from the group consisting of an antibody or antigen binding fragment thereof, an antibody domain, an antigen, a T-cell receptor, a cell surface receptor, a cell surface adhesion molecule, a major histocompatibility locus protein, a viral envelope protein and a peptide selected by phage display that binds specifically to a defined cell.

Targeting domains to specific cells can be accomplished by fusing directly, via a linker peptide sequence, or chemically conjugating the sequence to a di-/multimerization polypeptide, or to a hydrophobic or insoluble tail. These sequences target specific cells and tissues, but in some embodiments the interaction of the targeting signal with the cell does not occur through a traditional receptor:ligand interaction. The eukaryotic cell includes a number of distinct cell surface molecules. The structure and function of each molecule can be specific to the origin, expression, character and structure of the cell. Determining the unique cell surface complement of molecules of a specific cell type can be determined using techniques well known in the art.

It is known in the art that nearly every cell type in a tissue in a mammalian organism possesses some unique cell surface receptor or antigen. Thus, it is possible to incorporate nearly any ligand for the cell surface receptor or antigen as a targeting signal. For example, peptidyl hormones can be used as targeting moieties to target delivery to those cells which possess receptors for such hormones. Chemokines and cytokines can similarly be employed as targeting signals to target delivery of the complex to their target cells. A variety of technologies have been developed to identify genes that are preferentially expressed in certain cells or cell states and one of skill in the art can employ such technology to identify targeting signals which are preferentially or uniquely expressed on the target tissue of interest.

a. Brain Targeting

In one embodiment, the targeting signal is directed to cells of the nervous system, including the brain and peripheral nervous system. Cells in the brain include several types and states and possess unique cell surface molecules specific for the type. Furthermore, cell types and states can be further characterized and grouped by the presentation of common cell surface molecules.

In one embodiment, the targeting signal is directed to specific neurotransmitter receptors expressed on the surface of cells of the nervous system. The distribution of neurotransmitter receptors is well known in the art and one so skilled can direct the compositions described by using neurotransmitter receptor specific antibodies as targeting signals. Furthermore, given the tropism of neurotransmitters for their receptors, in one embodiment the targeting signal consists of a neurotransmitter or ligand capable of specifically binding to a neurotransmitter receptor.

In one embodiment, the targeting signal is specific to cells of the nervous system which may include astrocytes, microglia, neurons, oligodendrites and Schwann cells. These cells can be further divided by their function, location, shape, neurotransmitter class and pathological state. Cells of the nervous system can also be identified by their state of differentiation, for example stem cells. Exemplary markers specific for these cell types and states are well known in the art and include, but are not limited to CD133 and Neurosphere.

b. Muscle Targeting

In one embodiment, the targeting signal is directed to cells of the musculoskeletal system. Muscle cells include several types and possess unique cell surface molecules specific for the type and state. Furthermore, cell types and states can be further characterized and grouped by the presentation of common cell surface molecules.

In one embodiment, the targeting signal is directed to specific neurotransmitter receptors expressed on the surface of muscle cells. The distribution of neurotransmitter receptors is well known in the art and one so skilled can direct the compositions described by using neurotransmitter receptor specific antibodies as targeting signals. Furthermore, given the tropism of neurotransmitters for their receptors, in one embodiment the targeting signal consists of a neurotransmitter. Exemplary neurotransmitters expressed on muscle cells that can be targeted include but are not limited to acetylcholine and norepinephrine.

In one embodiment, the targeting signal is specific to muscle cells which consist of two major groupings, Type I and Type II. These cells can be further divided by their function, location, shape, myoglobin content and pathological state. Muscle cells can also be identified by their state of differentiation, for example muscle stem cells. Exemplary markers specific for these cell types and states are well known in the art include, but are not limited to MyoD, Pax7 and MR4.

c. Antibodies

Another embodiment provides an antibody or antigen binding fragment thereof acting as the targeting signal. The antibodies or antigen binding fragment thereof are useful for directing the protein vesicles to a cell type or cell state. In one embodiment, the protein vesicles possess an antibody binding domain, for example from proteins known to bind antibodies such as Protein A and Protein G from Staphylococcus aureus. Therefore, in some embodiments the protein vesicles include an antibody binding domain from Protein A or Protein G. Other domains known to bind antibodies are known in the art and can be substituted. In certain embodiments, the antibody is polyclonal, monoclonal, linear, humanized, chimeric or a fragment thereof. Representative antibody fragments are those fragments that bind the antibody binding portion of the non-viral vector and include Fab, Fab′, F(ab′), Fv diabodies, linear antibodies, single chain antibodies and bispecific antibodies known in the art.

In some embodiments, the targeting domain includes all or part of an antibody that directs the protein vesicle to the desired target cell type or cell state. Antibodies can be monoclonal or polyclonal, but are preferably monoclonal. Antibodies can be derived from human genes and specific for cell surface markers, and can be produced to reduce potential immunogenicity to a human host as is known in the art. For example, transgenic mice which contain the entire human immunoglobulin gene cluster are capable of producing “human” antibodies can be utilized. In one embodiment, fragments of such human antibodies are employed as targeting signals. In a preferred embodiment, single chain antibodies modeled on human antibodies are prepared in prokaryotic culture.

An exemplary class of targeting signals or domains is cancer targeting protein or peptide. In a preferred embodiment, monoclonal antibody is used as cancer targeting agents, including the antibody and radiolabeled antibody. In another preferred embodiment, cancer targeting peptide serves as the hydrophilic headgroup as a building block to make protein vesicles. Examples of cancer cell surface peptides, antibodies or polypeptide identified to be effective in targeting cancer cell surface peptides, and methods known in the art to identify targeting peptides are disclosed in Aina O H et al., Biopolymers, 66(3):184-99 (2002), which is incorporated by reference herein.

Various types of antibodies and antibody fragments can be used in the described compositions and methods, including whole immunoglobulin of any class, fragments thereof, and synthetic proteins containing at least the antigen binding variable domain of an antibody. The antibody can be an IgG antibody, such as IgG₁, IgG₂, IgG₃, or IgG₄. An antibody can be in the form of an antigen binding fragment including a Fab fragment, F(ab′)2 fragment, a single chain variable region, and the like. Antibodies can be polyclonal or monoclonal (mAb). Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison, et al., Proc. Natl. Acad. Sci. USA, 81: 6851-6855 (1984)). The described antibodies can also be modified by recombinant means, for example by deletions, additions or substitutions of amino acids, to increase efficacy of the antibody in mediating the desired function. Substitutions can be conservative substitutions. For example, at least one amino acid in the constant region of the antibody can be replaced with a different residue (see, e.g., U.S. Pat. No. 5,624,821; U.S. Pat. No. 6,194,551; WO 9958572; and Angal, et al., Mol. Immunol. 30:105-08 (1993)). In some cases changes are made to reduce undesired activities, e.g., complement-dependent cytotoxicity. The antibody can be a bi-specific antibody having binding specificities for at least two different antigenic epitopes. In one embodiment, the epitopes are from the same antigen. In another embodiment, the epitopes are from two different antigens. Bi-specific antibodies can include bi-specific antibody fragments (see, e.g., Hollinger, et al., Proc. Natl. Acad. Sci. U.S.A., 90:6444-48 (1993); Gruber, et al., J. Immunol., 152:5368 (1994)).

Antibodies can be generated by any means known in the art. Exemplary descriptions means for antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); and Current Protocols In Immunology (John Wiley & Sons, most recent edition). Fragments of intact Ig molecules can be generated using methods well known in the art, including enzymatic digestion and recombinant means.

2. Imaging Proteins

In some embodiments, the protein vesicles include fluorescent or luminescent proteins to provide the ability to visualize, track, and quantify molecules and events in biological systems. In a preferred embodiment, fluorescent or luminescent proteins are fused or conjugated with another polypeptide as a building block for protein vesicles. Exemplary fluorescent proteins include green fluorescent protein (GFP), enhanced green fluorescent protein (EGFP), mCherry, blue fluorescent protein (BFP), cyan fluorescent protein (CFP), and fusions or variations thereof, and are disclosed in Lippincott-Schwartz and Patterson, Science, 300 (5616): 87-91 (2003) which is incorporated by reference herein. Other fluorescent and luminescent proteins are known in the art or their sequences are available from GenBank. They are also available from conventional vendors including ThermoFisher, Clontech, Sigma.

In a preferred embodiment, the fluorescent protein has at least 80, 85, 90, 95, 99, or 100 percent sequence identity to that of mCherry:

| (SEQ ID NO: 2) | |
|---|---|
| MVSKGEEDNM AIIKEFMRFK VHMEGSVNGH EFEIEGEGEG RPYEGTQTAK LKVTKGGPLP | 60 |
| FAWDILSPQF MYGSKAYVKH PADTPDYLKL SFPEGFKWER VMNFEDGGVV TVTQDSSLQD | 120 |
| GEFIYKVKLR GTNEPSDGPV MUKTMGWER SSERMYPEDG ALKGEIKQRL KLKDGGHYDA | 180 |
| FVKTTYKAKK PVQLPGAYNV NIKLDITSHN EDYTIVEQYE RAFGRHSTGG MDELYK. | |

In another embodiment, a GFP or its variant is encoded by a nucleic acid having at least 80, 85, 90, 95, 97, 99, or 100% sequence identity to that of a wild-type GFP:

| (SEQ ID NO: 3) | |
|---|---|
| MSKGEELFTG VVPILVELDG DVNGHKFSVS GEGEGDATYG KLTLKFICTT GKLPVPWPTL | 60 |
| VTIFSYGVQC FSRYPDHMKQ HDFFKSAMPE GYVQERTIFF KDDGNYKTRA FVKFEGDTLV | 120 |
| NRIELKGTDF KEDGNILGHK LEYNYNSHNV YIMADKQKNG IKVNEKIRHN IEDGSVQLAD | 180 |
| HYQQNTPIGD GPVELPDNEY LSTQSALSXD PNEKRDHMVL LEFVIAAGIT HGMDELYK. | |

In still another embodiment, an EGFP or its variant is encoded by a nucleic acid having at least 80, 85, 90, 95, 97, 99, or 100% sequence identity to that of:

| (SEQ ID NO: 4, GenBank Accession No. JQ064510.1, Synthetic construct | |
|---|---|
| clone eGFP-OsP5SM_E/R eGFP (eGFP) gene, complete cds) | |
| atgtctagag tgagcaaggg cgaggagctg ttcaccgggg ttgttcccat cctggtcgag | 60 |
| ctggacggcg acgtaaacgg ccacaagttc agcgtgtccg gcgagggcga gggcgatgcc | 120 |
| acctacggca agctgaccct gaagttcatc tgcaccaccg gcaagctgcc cgtgccctgg | 180 |
| cccaccctcg tgaccaccct gacctacggc gtgcagtgct tcagccgcta ccccgaccac | 240 |
| atgaagcagc acgacttctt caagtccgcc atgcccgaag gctacgtcca ggaggtagat | 300 |
| ttatgcatcc tcttgtcatg agaagtcgaa ttgttcccat tctgtgtgtt gcagctacag | 360 |
| atagagatac atagagatac tcgtggattt tgcttagtgt tgagttttgt tctggttgtg | 420 |
| aactaaaagt ttatacattt gcaggaaata aatagccttt tgtttaaatc aaaaggtctt | 480 |
| acctatgtta gtgtgaagca ttggatccca aagaactcca aaatgcgatg aggcatattt | 540 |
| aatcttgtct ggactagtaa caggttggga tgaccacctg tgaagctcca acaggattgc | 600 |
| ctcctcacgc aatgtttgag gtctgatgtt caatagcttg ttttgtttca ctttgctttg | 660 |
| gactttcttt tcgccaatga gctatgtttc tgatggtttt cactcttttg gtgtgtagag | 720 |
| aaccatcttc ttcaaggacg acggcaacta caagacccgc gccgaggtga agttcgaggg | 780 |
| cgacaccctg gtgaaccgca tcgagctgaa gggcatcgac ttcaaggagg acggcaacat | 840 |
| cctggggcac aagctggagt acaactacaa cagccacaac gtctatatca tggccgacaa | 900 |
| gcagaagaac ggcatcaagg tgaacttcaa gatccgccac aacatcgagg acggcagcgt | 960 |
| gcagctcgcc gaccactacc agcagaacac ccccatcggc gacggccccg tgctgctgcc | 1020 |
| cgacaaccac tacctgagca cccagtccgc cctgagcaaa gaccccaacg agaagcgcga | 1080 |
| tcacatggtc ctgctggagt tcgtgaccgc cgccgggatc actctcggca tggacgagct | 1140 |
| gtacaagtaa | 1150 |

3. Enzymes

The fusion proteins or bonded proteins, in some embodiments, contain an amino acid sequence corresponding to enzymes or fragments thereof. Enzymes are biological catalysts and can serve as a hydrophilic headgroup in a fusion protein to make protein vesicles.

In some embodiments, the enzymes are proteases that break down proteins, starches, fats and grease. Examples of these enzymes include, but are not limited to, amylases; lipases; oxidases such as glucose oxidase, sorbitol oxidase, choline oxidase, hexose oxidase, and alcohol oxidase; hemicellulases, peroxidases, proteases, cellulases, xylanases, lipases, phospholipases, esterases, cutinases, pectinases, pectate lyases, keratinases, reductases, oxidases, oxido reductases, phenoloxidases, lipoxygenases, ligninases, mannanases, pullulanases, tannases, pentosanases, peroxidases, malanases, β-glucanases, arabinosidases, hyaluronidase, chondroitinase, laccase, endoglycosidases; or fragments thereof.

In some embodiments, the enzymes are therapeutic biocatalysts. Examples of these enzymes include, but are not limited to, prolactazyme, beta-lactamase, aglucerase, streptokinase, asparaginase, collagenase, DNAse, lysozyme, ribonuclease, trypsin, and uricase. Specific examples of these enzymes are known in the art.

4. Therapeutic Agents

Therapeutic agents can serve as a hydrophilic headgroup in a building block of the protein vesicles. These therapeutic agents include thyroid stimulating hormone; beneficial lipoproteins such as Apo1; prostacyclin and other vasoactive substances, anti-oxidants and free radical scavengers; soluble cytokine receptors, for example soluble transforming growth factor (TGF) receptor, or cytokine receptor antagonists, for example IL1ra; soluble adhesion molecules, for example ICAM-1; soluble receptors for viruses, e.g. CD4, CXCR4, CCR5 for HIV; cytokines; elastase inhibitors; bone morphogenetic proteins (BMP) and BMP receptors 1 and 2; endoglin; serotonin receptors; tissue inhibiting metaloproteinases; potassium channels or potassium channel modulators; anti-inflammatory factors; angiogenic factors including vascular endothelial growth factor (VEGF), transforming growth factor (TGF), hepatic growth factor, and hypoxia inducible factor (HIF); polypeptides with neurotrophic and/or anti-angiogenic activity including ciliary neurotrophic factor (CNTF), glial-derived neurotrophic factor (GDNF), nerve growth factor (NGF), brain-derived neurotrophic factor (BDNF), neurotrophin-3, nurturin, fibroblast growth factors (FGFs), endostatin, ATF, fragments of thrombospondin, variants thereof and the like. More preferred polypeptides are FGFs, such as acidic FGF (aFGF), basic FGF (bFGF), FGF-1 and FGF-2 and endostatin.

In some embodiments, the active agent is a protein or peptide. Examples of protein active agents include, but are not limited to, cytokines and their receptors, as well as chimeric proteins including cytokines or their receptors, including, for example tumor necrosis factor alpha and beta, their receptors and their derivatives; renin; lipoproteins; colchicine; prolactin; corticotrophin; vasopressin; somatostatin; lypressin; pancreozymin; leuprolide; alpha-1-antitrypsin; clotting factors such as factor VIIIC, factor IX, tissue factor, and von Willebrands factor; anti-clotting factors such as Protein C; atrial natriuretic factor; lung surfactant; a plasminogen activator other than a tissue-type plasminogen activator (t-PA), for example a urokinase; bombesin; thrombin; hemopoietic growth factor; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; a microbial protein, such as beta-lactamase; DNase; inhibin; activin; receptors for hormones or growth factors; integrin; protein A or D; rheumatoid factors; platelet-derived growth factor (PDGF); epidermal growth factor (EGF); transforming growth factor (TGF) such as TGF-α, and TGF-β, including TGF-β1, TGF-β2, TGF-Jβ3, TGF-β4, or TGF-β5; insulin-like growth factor-I and -II (IGF-I and IGF-II); des(1-3)-IGF-I (brain IGF-1), insulin-like growth factor binding proteins; CD proteins such as CD-3, CD-4, CD-8, and CD-19; erythropoietin; osteoinductive factors; immunotoxins; an interferon such as interferon-alpha (e.g., interferon.alpha.2A), -beta, -gamma, -lambda and consensus interferon; colony stimulating factors (CSFs), e.g., M-CSF, GM-CSF, and G-CSF; interleukins (ILs), e.g., IL-I to IL-10; superoxide dismutase; T-cell receptors; surface membrane proteins; decay accelerating factor; transport proteins; homing receptors; addressins; fertility inhibitors such as the prostaglandins; fertility promoters; regulatory proteins; antibodies (including fragments thereof) and chimeric proteins, such as immunoadhesins; precursors, derivatives, prodrugs and analogues of these compounds, and pharmaceutically acceptable salts of these compounds, or their precursors, derivatives, prodrugs and analogues. Suitable proteins or peptides may be native or recombinant and include, e.g., fusion proteins.

Hormones to be included in the disclosed products or, most preferably, produced from cells included in the disclosed products can be any hormone of interest.

Examples of endocrine hormones include anti-diuretic hormone (ADH), which is produced by the posterior pituitary, targets the kidneys, and affects water balance and blood pressure; oxytocin, which is produced by the posterior pituitary, targets the uterus, breasts, and stimulates uterine contractions and milk secretion; growth hormone (GH), which is produced by the anterior pituitary, targets the body cells, bones, muscles, and affects growth and development; prolactin, which is produced by the anterior pituitary, targets the breasts, and maintains milk secretions; growth hormone-releasing hormone (GHRH), which is a releasing hormone of GH and is produced in the arcuate nucleus of the hypothalamus; thyroid stimulating hormone (TSH), which is produced by the anterior pituitary, targets the thyroid, and regulates thyroid hormones; thyrotropin-release hormone (TRH), which is produced by the hypothalamus and stimulates the release of TSH and prolactin from the anterior pituitary; adrenocorticotropic hormone (ACTH), which is produced by the anterior pituitary, targets the adrenal cortex, and regulates adrenal cortex hormones; follicle-stimulating hormone (FSH), which is produced by the anterior pituitary, targets the ovaries/testes, and stimulates egg and sperm production; lutenizing hormone (LH), which is produced by the anterior pituitary, targets the ovaries/testes, and stimulates ovulation and sex hormone release; luteinizing hormone-releasing hormone (LHRH), also known as gonadotropin-releasing hormone (GnRH), which is synthesized and released from GnRH neurons within the hypothalamus and is a trophic peptide hormone responsible for the release of FSH and LH; Thyroxine, which is produced by the thyroid, targets the body cells, and regulates metabolism; calcitonin, which is produced by the thyroid, targets the adrenal cortex, and lowers blood calcium; parathyroid hormone, which is produced by the parathyroid, targets the bone matrix, and raises blood calcium; aldosterone, which is produced by the adrenal cortex, targets the kidney, and regulates water balance; cortisol, which is produced by the adrenal cortex, targets the body cells, and weakens immune system and stress responses; Epinephrine, which is produced by the adrenal medulla, targets the heart, lungs, liver, and body cells, and affects primary “fight or flight” responses; glucagon, which is produced by the pancreas, targets the liver body, and raises blood glucose level; insulin, which is produced by the pancreas, targets body cells, and lowers blood glucose level; Estrogen, which is produced by the ovaries, targets the reproductive system, and affects puberty, menstrual, and development of gonads; progesterone, which is produced by the ovaries, targets the reproductive system, and affects puberty, menstrual cycle, and development of gonads; and testosterone, which is produced by the adrenal gland, testes, targets the reproductive system, and affects puberty, development of gonads, and sperm.

In some embodiments, the protein is a growth hormone, such as human growth hormone (hGH), recombinant human growth hormone (rhGH), bovine growth hormone, methione-human growth hormone, des-phenylalanine human growth hormone, and porcine growth hormone; insulin, insulin A-chain, insulin B-chain, and proinsulin; or a growth factor, such as vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and (IGF-I and IGF-II).

B. Di-/Multimerization Domains to Associate Hydrophilic Protein Head and Hydrophobic Tail

Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric.

1, Dimerization Domains

a. Leucine Zipper Pairs

A “dimerization domain” is formed by the association of at least two amino acid residues or of at least two peptides or polypeptides (which may have the same, or different, amino acid sequences). The peptides or polypeptides may interact with each other through covalent and/or non-covalent association(s).

In a preferred embodiment, the dimerization domains are units of the heterodimeric leucine zipper pair. An arginine-rich leucine zipper motif (Z_{R}) is a domain in one population of fusion proteins or chemically linked protein moieties, and the glutamic acid-rich leucine zipper motif (Z_{E}) is a domain in another population of fusion proteins or chemically linked protein moieties. The two motifs coil into (i.e., form) leucine zippers.

Leucine zippers have the general structural formula known as the heptad repeat of a sequence Leucine-X₁-X₂-X₃-X₄-X₅-X₆ (SEQ ID NO: 5), where X may be any of the conventional 20 amino acids (Proteins, Structures and Molecular Principles, (1984) Creighton (ed.), W. H. Freeman and Company, New York, which is incorporated herein by reference), but are most likely to be amino acids with high α-helix forming potential, for example, alanine, valine, aspartic acid, glutamic acid, and lysine (Richardson and Richardson, Science 240:1648 (1988)), and the number of repeating sequence (n) may be 3 or greater, although typically n is 4 or 5. When n is 3, the specific leucine zipper has the formula of Leucine-X₁-X₂-X₃-X₄-X₅-X₆-Leucine-X₇-X₈-X₉-X₁₀-X₁₁-X₁₂-Leucine-X₁₃-X₁₄-X₁₅-X₁₆-X₁₇-X₁₈ (SEQ ID NO:6). When n is 4, the specific leucine zipper has the formula of Leucine-X₁-X₂-X₃-X₄-X₅-X₆-Leucine-X₇-X₈-X₉-X₁₀-X₁₁-X₁₂-Leucine-X₁₃-X₁₄-X₁₅-X₁₆-X₁₇-X₁₈-Leucine-X₁₉-X₂₀-X₂₁-X₂₂-X₂₃-X₂₄ (SEQ ID NO:7). When n is 5, the specific leucine zipper has the formula of Leucine-X₁-X₂-X₃-X₄-X₅-X₆-Leucine-X₇-X₈-X₉-X₁₀-X₁₁-X₁₂-Leucine-X₁₃-X₁₄-X₁₅-X₁₆-X₁₇-X₁₈-Leucine-X₁₉-X₂₀-X₂₁-X₂₂-X₂₃-X₂₄-Leucine-X₂₅-X₂₆-X₂₇-X₂₈-X₂₉-X₃₀ (SEQ ID NO:8). The 20 conventional amino acids (and their corresponding three letter code and one letter code) are: glycine (Gly, G), proline (Pro, P), lysine (Lys, K), arginine (Arg, R), histidine (His, H), methionine (Met, M), tryptophan (Trp, W), phenylalanine (Phe, F), isoleucine (Ile, I), leucine (Leu, L), valine (Val, V), alanine (Ala, A), serine (Ser, S), threonine (Thr, T), cysteine (Cys, C), glutamine (Gin, Q), asparagine (Asn, N), tyrosine (Tyr, Y), aspartic acid (Asp, D), and glutamic acid (Glu, E).

The leucine zippers have pairwise affinity. Leucine zippers form amphipathic alpha helices, and more specifically form coiled coils. Pairwise affinity is defined as the capacity for one species of leucine zipper, for example but not for limitation, the Fos leucine zipper, to predominantly form heterodimers with another specie of leucine zipper, for example but not for limitation, the Jun leucine zipper, such that heterodimer formation is preferred over homodimer formation when two species of leucine zipper are present in sufficient concentrations (U.S. Pat. No. 5,932,448, incorporated by reference in its entirety). Thus, predominant formation of heterodimers leads to a dimer population that is typically 50 to 75 percent, preferentially 75 to 85 percent, and most preferably more than 85 percent heterodimers.

A representative leucine zipper pair has the following amino acid sequences, where Z_{E} being:

(SEQ ID NO: 9)
LEIEAAALEQ ENTALETEVA ELEQEVQRLE NIVSQYRTRY GPL
and
Z_{R} being:
(SEQ ID NO: 10)
LEIRAAALRR RNTALRTRVA ELRQRVQRLR NEVSQYETRY GPL.

Those of skill in the art will understand that leucine zippers of the present invention may comprise amino acids sequences that are not identical to those shown, by, for example but not for limitation, having internal or terminal additions, deletions, or substitutions, or by having rearrangement of the order of the heptad repeats. Additional examples of the sequences of leucine zipper pairs are included in Moll J R, et al., Protein Sci., 10(3), 649-655 (2001), which is hereby incorporated by reference in its entirety. For illustration, but not for limitation, the invention encompasses additions or substitutions of terminal amino acids that comprise glycine and/or cysteine.

b. Disulfide

In another embodiment, dimerization domains contain at least one cysteine that is capable of forming an intermolecular disulfide bond with a cysteine on the partner fusion protein. The dimerization domain can contain one or more cysteine residues such that disulfide bond(s) can form between the partner fusion proteins. In one embodiment, dimerization domains contain one, two or three to about ten cysteine residues. This embodiment also allows for pH sensitive, reversible dimerization.

c. Others

Additional exemplary dimerization domain can be any known in the art and include, but not limited to, other coiled coils (e.g., tryptophan-zipper, see Liu J, et al., Proc. Nat. Acad. Sci. USA., 101, 46, 16156-16161 (2004)), acid patches, zinc fingers, calcium hands, hinge region of an immunoglobulin, a C_{H}1-C_{L} pair, an “interface” with an engineered “knob” and/or “protruberance” as described in U.S. Pat. No. 5,821,333, SH2 (src homology 2), SH3 (src Homology 3) (Vidal, et al., Biochemistry, 43, 7336-44 ((2004)), phosphotyrosine binding (PTB) (Zhou, et al., Nature, 378:584-592 (1995)), WW (Sudol, Prog. Biochys. Mol. Bio., 65:113-132 (1996)), PDZ (Kim, et al., Nature, 378: 85-88 (1995); Komau, et al., Science, 269:1737-1740 (1995)) 14-3-3, WD40 (Hu, et al., J Biol Chem., 273, 33489-33494 (1998)), an isoleucine zipper, a receptor dimer pair (e.g., interleukin-8 receptor (IL-8R); and integrin heterodimers such as LFA-1 and GPIIIb/IIIa), or the dimerization region(s) thereof, dimeric ligand polypeptides (e.g. nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa, et al., J. Biol. Chem., 269(45): 27833-27839 (1994) and Radziejewski, et al., Biochem., 32(48): 1350 (1993)) and can also be variants of these domains in which the affinity is altered. The polypeptide pairs can be identified by methods known in the art, including yeast two hybrid screens. Yeast two hybrid screens are described in U.S. Pat. Nos. 5,283,173 and 6,562,576. Affinities between a pair of interacting domains can be determined using methods known in the art, including as described in Katahira, et al., (J. Biol. Chem., 277, 9242-9246 (2002)). Alternatively, a library of peptide sequences can be screened for heterodimerization, for example, using the methods described in WO 01/00814. Useful methods for protein-protein interactions are also described in U.S. Pat. No. 6,790,624.

One embodiment provides an engineered coiled coil dimerization domain found in Zaccai, N. R., et al., Nature Chemical Biology, 7(12):935-941 (2011).

2. Multimerization Domains

A “multimerization domain” is a domain that causes three or more peptides or polypeptides to interact with each other through covalent and/or non-covalent association(s). Suitable multimerization domains include, but are not limited to, coiled-coil domains. A coiled-coil is a peptide sequence with a contiguous pattern of mainly hydrophobic residues spaced 3 and 4 residues apart, usually in a sequence of seven amino acids (heptad repeat) or eleven amino acids (undecad repeat), which assembles (folds) to form a multimeric bundle of helices. Coiled-coils with sequences including some irregular distribution of the 3 and 4 residues spacing are also contemplated. Hydrophobic residues are in particular the hydrophobic amino acids Val, Ile, Leu, Met, Tyr, Phe and Trp. “Mainly hydrophobic” means that at least 50% of the residues must be selected from the mentioned hydrophobic amino acids.

The coiled coil domain may be derived from laminin. In the extracellular space, the heterotrimeric coiled coil protein laminin plays an important role in the formation of basement membranes. Apparently, the multifunctional oligomeric structure is required for laminin function. Coiled coil domains may also be derived from the thrombospondins in which three (TSP-1 and TSP-2) or five (TSP-3, TSP-4 and TSP-5) chains are connected, or from COMP (COMPcc) (Guo, et al., EMBO J., 1998, 17: 5265-5272) which folds into a parallel five-stranded coiled coil (Malashkevich, et al., Science, 274: 761-765 (1996)).

Additional coiled-coil domains derived from other proteins, and other domains that mediate polypeptide multimerization are known in the art and are suitable for use in the disclosed fusion proteins.

In another embodiment, fusion proteins, or fragments thereof can be induced to form multimers by binding to a second multivalent polypeptide.

C. Moieties Serving as Hydrophobic or Insoluble Tail

1. Elastin-Like Peptide

In a preferred embodiment, an elastin-like peptide serves as the hydrophobic, insoluble tail. Elastin-like polypeptides (ELPs) are a class of temperature responsive biopolymers that are derived from a structural motif found in mammalian elastin (Gray W R, et al., Nature, 246(5434), 461-466 (1973); Tatham A S, et al., Trends Biochem. Sci., 25(11), 567-571 (2000)). This family of polypeptides comprises polymers of the pentapeptide of SEQ ID NO: 1, where the “guest residue” Xaa is any amino acid except Pro. Xaa can be the same or different in each repeat of SEQ ID NO: 1. In some embodiments, an ELP is composed of at least ten, twenty, thirty, forty, fifty, sixty, or more repeats of SEQ ID NO: 1. A representative elastin-like peptide has the following amino acid sequence:

(SEQ ID NO: 11)
VPGVGVPGVG VPGFGVPGVG VPGVG.

ELPs undergo an inverse temperature phase transition, also known as a lower critical solution temperature transition, in aqueous solution in response to an increase in solution temperatures (Li, et al., J. Am. Chem. Soc., 123(48), 11991-11998 (2001); Urry, Prog. Biophys. Mol. Biol., 57(1), 23-57 (1992); and Urry, J. Phys. Chem. B, 101(51), 11007-11028 (1997)). Below their transition temperature (Tₜ) they are soluble in aqueous solution. Above their Tₜ, however, ELPs undergo a sharp phase transition (˜2° C.) during which they hydrophobically collapse and aggregate. This phase transition is fully reversible, so that the aggregated ELP dissolves in aqueous solution once the temperature is decreased below the Tₜ. As a result, an ELP is also referred to herein as a “thermally responsive polypeptide” and/or “temperature-sensitive polypeptide”.

Representative Tₜs for in vitro applications include 0° C., 10° C., 15° C., 20° C. to 60° C., and 60° C. to 100° C., including all temperatures in between. For in vivo applications, representative Tₜs include 35° C. to 65° C. inclusive, including, but not limited to 35-40° C., 40-45° C., 45-50° C., 50-55° C., and 55-60° C.

One of skill in the art can employ an ELP having a certain Tₜ based upon such parameters as what temperatures the ELP is to be exposed to and whether it would be desirable for the ELP to remain soluble or become insoluble under specific conditions. In some embodiments, the Tₜ can be tuned by adjusting one or more of the guest residues (Xaa of SEQ ID NO: 1), the molecular weight (MW, or length of the ELP), and the ELP concentration (Meyer and Chilkoti, Biomacromolecules, 3(2), 357-367 (2002); Meyer and Chilkoti, Biomacromolecules, 5(3), 846-851 (2004)). For example, generally, as the hydrophobicity of the guest residue increases, the Tₜ decreases. Thus, for ELPs that include polymers of SEQ ID NO: 1, as the mole fraction of guest residues that are hydrophobic increases, the Tₜ of the ELP decreases. As such, ELPs can be synthesized with different Tₜs based upon the mole fraction of different residues chosen as the guest residue. The relative hydrophobicities and hydrophilities of the naturally occurring amino acids are known, as well as the general effect on Tₜ that can be expected when a given amino acid is present as the guest residue. The hierarchy of guest residues from most hydrophobic (that is, having the largest lowering effect on Tₜ) to least hydrophobic is Trp>Tyr>Phe>Leu>Ile>Met>Val>Cys>Ala>Thr>Asn>Ser>Gly>Arg>Gln>Lys (Urry, et al., J. Am. Chem. Soc., 113(11), 4346-4344 (1991); Urry, et al., J. Phys Chem. B., 101(51), 11007-11028 (1997)).

Additionally, a longer ELP will have a lower Tₜ than a shorter ELP with the same mole fraction of various guest residues. Thus, another way to influence the Tₜ of a given ELP is to lengthen or shorten it. For a given mole fraction of individual guest residues, the Tₜ can be varied over 20° C. or more depending on the length of the ELP. An example of this effect is described by Meyer and Chilkoti (Biomacromolecules, 3(2), 357-367 (2002)), where for an ELP with only Val, Ala, and Gly guest residues in a ratio of 5:2:3, respectively, a 60 amino acid ELP had a Tt of about 62° C. (25 μM in PBS), a 90 amino acid ELP has a Tt of about 50° C., a 150 amino acid ELP has a Tt of about 42° C., a 240 amino acid ELP has a Tₜ of about 38° C., and a 330 amino acid ELP has a Tₜ of about 36° C. In the same study, an ELP with only Val, Ala, and Gly guest residues in a ratio of 1:8:7, respectively, a 128 amino acid ELP has a Tₜ of about 77° C. (25 μM in PBS), a 160 amino acid ELP has a Tₜ of about 71° C., a 256 amino acid ELP has a Tₜ of about 63° C., and a 320 amino acid ELP has a Tₜ of about 60° C. Thus, by manipulating the mole fraction of the guest residue and the length of the ELP polypeptide, ELPs with Tₜs between about 20° C. and 80° C. can be designed.

In some embodiments, the ELP can include an ELP block copolymer (ELP_{BC}). The ELPBCs can have a linear AB diblock architecture, formed by fusing an N-terminal ELP gene with a high Tₜ (Tₜ>90° C., termed ELP2) to a C-terminal ELP gene that has a much lower Tₜ (Tₜ≅40° C., termed ELP12). These ELP_{BC}s are highly soluble at a solution temperature below the Tₜ of both ELP blocks. However, upon an increase in solution temperature the ELP_{BC}s often self-assemble into a spherical micelle when the low Tₜ block undergoes its inverse temperature phase transition. The notation for the ELP_{BC}s includes an N-terminal ELP gene followed by its number of pentapeptides, then a C-terminal ELP gene and its corresponding number of pentapeptides. For example, ELP2-64,12-72 is an ELP_{BC} with 64 pentapeptides of an ELP2 gene at the N-terminus followed by 72 pentapeptides of ELP12 at the C-terminus.

2. Hydrophobic Peptide

In some embodiments, a hydrophobic domain of polypeptides or a polypeptide containing 60%, 70%, 80%, 90%, 95% or 100% of hydrophobic amino acids serve as the hydrophobic tail in the formation of protein vesicles. An exemplary amino acid sequence of hydrophobic peptide is: VLAVAVLAVA (SEQ ID NO:12).

In other embodiments, peptide motifs based on leucine zippers, human collagen, human elastin, and silkworm silk are building blocks for thermally responsive biopolymers, and become insoluble upon temperature change of the medium, and are described in Mackay J and Chilkoti A, International Journal of Hyperthermia, 24:483-495 (2008) which is hereby incorporated by reference.

D. Linker Domain

A fusion protein as a building block for making the protein vesicles can optionally contain a peptide or polypeptide linker domain that separates a first polypeptide from a second polypeptide or a second polypeptide from a third polypeptide.

Suitable peptide/polypeptide linker domains include naturally occurring or non-naturally occurring peptides or polypeptides. Peptide linker sequences are at least 2 amino acids in length. Preferably the peptide or polypeptide domains are flexible peptides or polypeptides. A “flexible linker” herein refers to a peptide or polypeptide containing two or more amino acid residues joined by peptide bond(s) that provides increased rotational freedom for two polypeptides linked thereby than the two linked polypeptides would have in the absence of the flexible linker. Such rotational freedom allows in particular the functional, hydrophilic headgroup protein/peptide moiety to access target/substrate(s) or to function more efficiently. Exemplary flexible peptides/polypeptides include, but are not limited to, the amino acid sequences Gly-Ser, Gly-Ser-Gly-Ser (SEQ ID NO:13), Ala-Ser, Gly-Gly-Gly-Ser (SEQ ID NO:14), (Gly₄-Ser)₃ (SEQ ID NO:15) and (Gly₄-Ser)₄ (SEQ ID NO: 16). In a preferred embodiment, the peptide linker is Gly-(Gly-Ser)₆-Gly (SEQ ID NO:17); and this linker is fused between one coil of a leucine zipper pair and the ELP, or between the functional protein and one coil of a leucine zipper pair.

In another embodiment, the linker domain contains the hinge region of an immunoglobulin. In a preferred embodiment, the hinge region is derived from a human immunoglobulin. Suitable human immunoglobulins that the hinge can be derived from include IgG, IgD and IgA. In a further embodiment, the hinge region is derived from human IgG. Amino acid sequences of immunoglobulin hinge regions and other domains are known in the art.

Additional flexible peptide/polypeptide sequences are well known in the art.

E. Additional Sequences

A fusion protein as a building block for making the protein vesicles can optionally include additional sequences or moieties, including, but not limited to linkers and purification tags.

In a preferred embodiment the purification tag is a polypeptide. Polypeptide purification tags are known in the art and include, but are not limited to His tags which typically include six or more, typically consecutive, histidine residues; FLAG tags, which typically include the sequence DYKDDDDK (SEQ ID NO:18); haemagglutinin (HA) for example, YPYDVP (SEQ ID NO:19); MYC tag for example ILKKATAYIL (SEQ ID NO:20) or EQKLISEEDL (SEQ ID NO:21). Methods of using purification tags to facilitate protein purification are known in the art and include, for example, a chromatography step wherein the tag reversibly binds to a chromatography resin.

Purifications tags can be N-terminal or C-terminal to the fusion protein. The purification tags N-terminal to the fusion protein are typically separated from the polypeptide of interest at the time of the cleavage in vivo. Therefore, purification tags N-terminal to the fusion protein can be used to remove the fusion protein from a cellular lysate following expression and extraction of the expression or solubility enhancing amino acid sequence, but cannot be used to remove the polypeptide of interest. Purification tags C-terminal to the fusion protein can be used to remove the polypeptide of interest from a cellular lysate following expression of the fusion protein, but cannot be used to remove the expression or solubility enhancing amino acid sequence. Purification tags that are C-terminal to the expression or solubility enhancing amino acid sequence can be N-terminal to, C-terminal to, or incorporated within the sequence of the polypeptide of interest.

In some embodiments, larger fusion partners, such as protein domains (e.g., chitin-binding domain) or proteins (e.g., cu-tinase, green fluorescent protein (GFP), glutathione-S-transferase (GST), intein, maltose binding protein (MBP), are used to promote folding, solubility, purification, labeling, chemical ligation, or immobilization of the recombinant protein. If desired, the fusion tag can be detached from the protein of interest by cleavage of a linker region with a site-specific protease, which does not cleave the protein of interest.

In some embodiments, the fusion proteins or bonded proteins, contain an amino acid sequence corresponding to high affinity binding proteins or fragments thereof, including, but not limited to, biotin, avidin, NeutrAvidin, and streptavidin, fibronectin to permit binding capability upon the formation of protein vesicles.

F. Agents to be Encapsulated

The hollow structure of the present invention, protein vesicles, permits the encapsulation cargo into the interior space of vesicles. The cargo is with multiple length scales: small molecules (˜10⁰ nm), biomacromolecules such as proteins (˜10¹ nm), and nanoparticles (˜10² nm).

1. Active Agents

In some embodiments, the protein vesicles contain in its void space therapeutic, prophylactic, neutraceutical and diagnostic agents. Any suitable agent may be used. These include organic compounds, inorganic compounds, proteins, polysaccharides, nucleic acids or other materials that can be incorporated using standard techniques.

a. Nucleic Acid

Active agents include synthetic and natural proteins (including enzymes, peptide-hormones, receptors, growth factors, antibodies, signaling molecules), and synthetic and natural nucleic acids (including RNA, DNA, anti-sense RNA, triplex DNA, inhibitory RNA (RNAi), and oligonucleotides), and biologically active portions thereof. Suitable active agents have a size greater than about 1,000 Da for small peptides and polypeptides. Nucleic acids are more typically listed in terms of base pairs or bases (collectively “bp”). Nucleic acids with lengths above about 10 bp are typically used in the present method. More typically, useful lengths of nucleic acids for probing or therapeutic use will be in the range from about 20 bp (probes; inhibitory RNAs, etc.) to tens of thousands of bp for genes and vectors. The active agents may also be hydrophilic molecules, preferably having a low molecular weight.

b. Proteins

Examples of useful proteins include hormones such as insulin and growth hormones including somatomedins. Examples of useful drugs include neurotransmitters such as L-DOPA, antihypertensives or saluretics such as Metolazone from Searle Pharmaceuticals, carbonic anhydrase inhibitors such as Acetazolamide from Lederle Pharmaceuticals, insulin like drugs such as glyburide, a blood glucose lowering drug of the sulfonylurea class, synthetic hormones such as Android F from Brown Pharmaceuticals and Testred® (methyltestosterone) from ICN Pharmaceuticals.

In some embodiments, the targeting domains, imaging proteins, enzymes and therapeutic proteins mentioned previously in this application are encapsulated in the protein vesicles.

c. Compounds

Representative anti-cancer agents include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), and combinations thereof. Other suitable anti-cancer agents include angiogenesis inhibitors including antibodies to vascular endothelial growth factor (VEGF) such as bevacizumab (AVASTIN®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (Nexavar®), erlotinib (Tarceva®), pazopanib, axitinib, and lapatinib; transforming growth factor-α or transforming growth factor-β inhibitors, and antibodies to the epidermal growth factor receptor such as panitumumab (VECTIBIX®) and cetuximab (ERBITUX®).

Under the Biopharmaceutical Classification System (BCS), drugs can belong to four classes: class I (high permeability, high solubility), class II (high permeability, low solubility), class III (low permeability, high solubility) or class IV (low permeability, low solubility). Suitable active agents also include poorly soluble compounds; such as drugs that are classified as class II or class IV compounds using the BCS. Examples of class II compounds include: acyclovir, nifedipine, danazol, ketoconazole, mefenamic acid, nisoldipine, nicardipine, felodipine, atovaquone, griseofulvin, troglitazone glibenclamide and carbamazepine. Examples of class IV compounds include: chlorothiazide, furosemide, tobramycin, cefuroxmine, and paclitaxel.

In some embodiments, the active agents include antibiotics, antineoplastic agents, anti-virals, antifungals, toxins (e.g. ricin), radionuclides (e.g. 1-131, Y-90, Sm-153), hormone antagonists (e.g. tamoxifen), platinum complexes (e.g. cisplatin), oligonucleotides (e.g. antisense oligonucleotides or silencing (siRNA) olidonucleotides sequences), chemotherapeutic nucleotide and nucleoside analogs (e.g. capecitabine, gemcitabine), boron containing compound (e.g. carborane), photodynamic agents (e.g. rhodamine 123), enediynes (e.g. calicheamicins), and camptothecins (e.g. CPT-11, SN-38, C9), and tyrosine kinase inhibitors (e.g. imatinib mesylate). In an exemplary embodiment for treating or preventing the establishment or growth of a tumor, the therapeutic agent is doxorubicin, a taxane, or cisplatin. In another embodiment for treating or preventing the establishment or growth of a bacterial infection, the therapeutic agent is a quinalone (e.g. levofloxacin), a macrolide (e.g. azithromycin), or a cephalosporin (e.g. cefuroxime) antibiotic. In an exemplary embodiment for treating or preventing the establishment or growth of a viral infection, the therapeutic agent is a reverse transcriptase inhibitor. In an exemplary embodiment for treating or preventing the establishment or growth of a fungal infection, the therapeutic agent is amphotericin B or nystatin. For the purposes of treating subjects having neoplastic disorders, the entrapped therapeutic agent is, in one embodiment, a cytotoxic drug. Cytotoxic agents are particularly useful as the entrapped agent in liposomes targeted for neoplastic disease indications. The drug may be an anthracycline antibiotic selected from doxorubicin, daunorubicin, epirubicin and idarubicin and analogs thereof. The cytotoxic drug can be a nucleoside analog selected from gemcitabine, capecitabine, and ribavirin. The cytotoxic agent may also be a platinum compound selected from cisplatin, carboplatin, ormaplatin, and oxaliplatin. The cytotoxic agent may be a topoisomcrasc 1 inhibitor selected from the group consisting of topotccan, irinotecan, SN-38, 9-aminocamptothecin and 9-nitrocamptothecin. The cytotoxic agent may be a vinca alkaloid selected from the group consisting of vincristine, vinblastine, vinleurosine, vinrodisine, vinorelbine and vindesine.

In another embodiment, the entrapped agent is useful for treating HIV infections and inhibiting HTV replication. The entrapped agent is selected from nucleoside HIV reverse transcriptase inhibitors, non-nucleoside HIV reverse transcriptase inhibitors, HIV protease inhibitors, HIV integrase inhibitors, HIV fusion inhibitors, immune modulators, CCR5 antagonists and antiinfectives is claimed. The nucleoside HIV reverse transcriptase inhibitors may be selected from abacavir, acyclovir, didanosine, emtricitabine, lamivudine, zidovudine, stavudine, atazanavir, and tenofovir. The non-nucleoside HIV reverse transcriptase inhibitors can be efavirenz, nevirapine, and calanolide. The HIV protease inhibitors can be amprenavir, nelfmavir, lopinavir, saquinavir, atazanavir, indinavir, tipranavir, and fosamprenavir calcium. The HIV fusion inhibitors can be enfuvirtide, T-1249, and AMD-3100. The CCR5 antagonists can be TAK-779, SC-351125, SCH-D, UK-427857, PRO-140, and GW-873140. Anti-HLA-DR coated liposomes containing the HTV protease inhibitor, indinavir, have been disclosed (Gagne et al. (2002) Biochim. Biophys. Acta 1558:198-210).

One aspect provides immune system modulators which are used as first line therapy or are given in conjunction (prior to, contemporaneously, or following) other types or therapeutic treatments especially for treatment of neoplastic disease and HIV infection or may be immunosuppressant drugs. The immune modulators may be chosen from an interferon (IFN) including an IFNalpha, IFNbeta or IFNgamma-type interferon; granulocyte macrophage colony stimulating factor (GM-CSF), granulocyte colony stimulating sactor (G-CSF), TNFalpha, and IL-2. The immuno-suppressant agents of the invention may be chosen from cyclosporine, sirolimus, and mycophenolate mofetil.

For imaging, radioactive materials such as Technetium99 (^{99m}Tc) or magnetic materials such as Fe₂O₃ could be used. Examples of other materials include gases or gas emitting compounds, which are radio opaque.

d. Vaccines

In some embodiments, the protein vesicles of are used to deliver antigens to stimulate a body's immune system, e.g. vaccine. The vesicles can display the antigen on the exterior surface of the vesicle or the vesicle can be loaded with the antigen. One embodiment provides adjuvants in the membrane or interior of the vesicle (depending on if their targets are on the cell surface, or inside). For example, TLR-5 adjuvant flagellin (a protein) can be on the surface of a vesicle as a headgroup, but CpG DNA TLR-9 adjuvant would be encapsulated inside the vesicle. Whether the antigen is inside or outside or both depends on the antigen/disease.

Preferred antigens can be presented at the surface of antigen presenting cells (APC) of a subject for surveillance by immune effector cells, such as leucocytes expressing the CD4 receptor (CD4 T cells) and Natural Killer (NK) cells. Typically, the antigen is of viral, bacterial, protozoan, fungal, or animal origin. In some embodiments the antigen is a cancer antigen. Cancer antigens can be antigens expressed only on tumor cells and/or required for tumor cell survival.

Certain antigens are recognized by those skilled in the art as immunostimulatory (i.e., stimulate effective immune recognition) and provide effective immunity to the organism or molecule from which they derive. Antigens can be peptides, proteins, polysaccharides, saccharides, lipids, nucleic acids, or combinations thereof. The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof. Suitable antigens are known in the art and are available from commercial government and scientific sources. The antigens may be purified or partially purified polypeptides derived from tumors or viral or bacterial sources. The antigens can be recombinant polypeptides produced by expressing DNA encoding the polypeptide antigen in a heterologous expression system. The antigens can be DNA encoding all or part of an antigenic protein. Antigens may be provided as single antigens or may be provided in combination. Antigens may also be provided as complex mixtures of polypeptides or nucleic acids.

A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3. Viral antigens may be derived from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

The antigen can be an allergen or environmental antigen, such as, but not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including i.a. birch (Betula), aider (Alnus), hazel (Corylus), hornbeam (Carpinus) and olive (Olea), cedar (Cryptomeria and Juniperus), Plane tree (Platanus), the order of Poales including i.e. grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

The antigen can be a tumor antigen, including a tumor-associated or tumor-specific antigen, such as, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARa fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, b-Catenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS.

2. Nanoparticles

In some embodiments, therapeutic or imaging nanoparticles are encapsulated in the protein vesicles. The nanoparticles may be formed from one or more polymers, copolymers, or polymer blends. In some embodiments, the one or more polymers, copolymers, or polymer blends are biodegradable. Examples of suitable polymers include, but are not limited to, polyhydroxyacids such as poly(lactic acid), poly(glycolic acid), and poly(lactic acid-co-glycolic acids); polycaprolactones; poly(orthoesters); polyanhydrides; poly(phosphazenes); poly(hydroxyalkanoates); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), poly(alkylene glycols) such as polyethylene glycol (PEG), poly(propylene glycol) (PPG), and copolymers of ethylene glycol and propylene glycol, poly(oxyethylated polyol), poly(olefinic alcohol), polyvinylpyrrolidone), poly(hydroxy alkylmethacrylamide), poly(hydroxyalkylmethacrylate), poly(saccharides), poly(vinyl alcohol), as well as blends and copolymers thereof. Techniques for preparing suitable polymeric nanoparticles are known in the art, and include solvent evaporation, hot melt particle formation, solvent removal, spray drying, phase inversion, coacervation, and low temperature casting. These nanoparticles can also be fluorescently labeled.

Small molecules, biologically active macromolecules (e.g., protein, peptide, and nucleic acid), or other active agents can be further incorporated into these nanoparticles for a controlled delivery. These molecules can be encapsulated, blended, covalently attached, or otherwise associated with the polymers prior to, during, or post formation of nanoparticles on the surface and/or in the interior of the nanoparticles.

Another embodiment provides encapsulate non-polymeric particles including, but not limited to inorganic nanoparticles, for example those made from silica, gold or other metals. The nanoparticles can be quantum dots.

G. Formulations

The vesicle compositions are provided in any suitable form, including for example, but are not limited to liquids, powder, granules, emulsions, gels, and pastes.

### Kits

In some embodiments, the compositions are provided in a kit. Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the protein vesicles and compositions encapsulated in the protein vesicles. The term “carrier” includes but is not limited to diluents, binders, lubricants, disintegrators, fillers, and coating compositions. “Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Baltimore, Md., 2000, and “Pharmaceutical dosage forms and drug delivery systems”, 6^{th} Edition, Ansel et. al., (Media, Pa.: Williams and Wilkins, 1995) which provides information on carriers, materials, equipment and process for preparing tablets and capsules and delayed release dosage forms of tablets, capsules, and granules.

In some additional embodiments, the enzyme delivering composition is suitable for use in cleaning applications, and the composition may also include one or more adjunct materials, dye transfer inhibiting agents, catalytic metal complexes, detergent builders or builder systems. Suitable adjunct materials include but are not limited to surfactants, builders, chelating agents, dye transfer inhibiting agents, deposition aids, dispersants, additional enzymes, and enzyme stabilizers, catalytic materials, bleach activators, bleach boosters, preformed peracids, polymeric dispersing agents, clay soil removal/anti-redeposition agents, brighteners, suds suppressors, dyes, perfumes, structure elasticizing agents, fabric softeners, carriers, hydrotropes, processing aids and/or pigments (See e.g., U.S. Pat. Nos. 5,576,282, 6,306,812, and 6,326,348, herein incorporated by reference). Suitable polymeric dye transfer inhibiting agents include, but are not limited to, polyvinylpyrrolidone polymers, polyamine N-oxide polymers, copolymers of N-vinylpyrrolidone and N-vinyl imidazole, polyvinyloxazolidones and polyvinylimidazoles or mixtures thereof. When present in a subject cleaning composition, the dye transfer inhibiting agents are typically present at levels from about 0.0001% to about 10%, from about 0.01% to about 5% or even from about 0.1% to about 3% by weight of the cleaning composition. In some yet further embodiments, the cleaning compositions also contain dispersants. Suitable water-soluble organic materials include the homo- or co-polymeric acids or their salts, in which the polycarboxylic acid comprises at least two carboxyl radicals separated from each other by not more than two carbon atoms.

### III. Methods of Preparing Protein Vesicles

A. Protein Expression

Fusion proteins can be obtained by, for example, chemical synthesis or by recombinant production in a host cell. To recombinantly produce a fusion protein, a nucleic acid containing a nucleotide sequence encoding the fusion protein can be used to transform, transduce, or transfect a bacterial or eukaryotic host cell (e.g., an insect, yeast, or mammalian cell). In general, nucleic acid constructs include a regulatory sequence operably linked to a nucleotide sequence encoding the fusion protein. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well known in the art include, for example, Escherichia coli strains such as BL-21, and cultured mammalian cells such as CHO cells.

In eukaryotic host cells, a number of viral-based expression systems can be utilized to express fusion proteins. Viral based expression systems are well known in the art and include, but are not limited to, baculoviral, SV40, retroviral, or vaccinia based viral vectors.

Mammalian cell lines that stably express variant fusion proteins can be produced using expression vectors with appropriate control elements and a selectable marker. For example, the eukaryotic expression vectors pCR3.1 (Invitrogen Life Technologies) and p91023(B) (see Wong et al. (1985) Science 228:810-815) are suitable for expression of variant costimulatory polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, a fusion protein can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Fusion proteins can be isolated using, for example, chromatographic methods such as DEAF ion exchange, gel filtration, and hydroxylapatite chromatography. For example, a costimulatory polypeptide in a cell culture supernatant or a cytoplasmic extract can be isolated using a protein G column. In some embodiments, fusion proteins can be engineered to contain an additional domain containing amino acid sequence that allows the polypeptides to be captured onto an affinity matrix. For example, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify costimulatory polypeptides. Fusion proteins can additionally be engineered to contain a secretory signal (if there is not a secretory signal already present) that causes the fusion protein to be secreted by the cells in which it is produced. The secreted fusion proteins can then conveniently be isolated from the cell media. In some embodiments, the compositions disclosed herein include expression of proteolysis resisting amino acid sequence.

In a preferred embodiment, Z_{R}-ELP was co-expressed with His₆-tagged Z_{E} to prevent degradation during expression. Co-expressed Z_{R}-ELP and His6-ZE form a protein complex His₆Z_{E}/Z_{R}-ELP, which is resistant to proteolysis in the cytoplasm.

In some embodiments, the compositions disclosed herein include expression of solubility enhancing amino acid sequence. In some embodiments, the expression or solubility enhancing amino acid sequence is cleaved prior to administration of the composition to a subject in need thereof. The expression or solubility enhancing amino acid sequence can be cleaved in the recombinant expression system, or after the expressed protein in purified. In some embodiments, the expression or solubility enhancing is a ULP1 or SUMO sequence. Recombinant protein expression systems that incorporate the SUMO protein (“SUMO fusion systems”) have been shown to increase efficiency and reduce defective expression of recombinant proteins in E. coli., see for example Malakhov, et al., J. Struct. Funct. Genomics, 5: 75-86 (2004), U.S. Pat. No. 7,060,461, and U.S. Pat. No. 6,872,551. SUMO fusion systems enhance expression and solubility of certain proteins, including severe acute respiratory syndrome coronavirus (SARS-CoV) 3CL protease, nucleocapsid, and membrane proteins (Zuo et al., J. Struct. Funct. Genomics, 6:103-111 (2005)).

B> Peptide Synthesis

Alternatively, some polypeptides are synthesized in a solid phase peptide synthesis process. Solid phase peptide synthesis is a known process in which amino acid residues are added to peptides that have been immobilized on a solid support.

C. Assembly of Protein Vesicles

The method of forming the protein vesicles includes mixing a first protein amphiphile or bonded polypeptide and a second protein amphiphile or bonded polypeptide together. In a preferred embodiment where elastin-like peptides are used as the hydrophobic tail, the mixing of two fused proteins or polypeptides are performed at a lower temperature, e.g. one that is below the lower critical transition temperature, Tₜ. In one embodiment, the method includes dissolving or dispersing a plurality of the first protein amphiphile or bonded polypeptide and a plurality of the second protein amphiphile or bonded polypeptide in an aqueous solution to form a heterogeneous dispersion or solution of polypeptides, wherein the first protein amphiphile or bonded polypeptide and second protein amphiphile or bonded polypeptide are preferably different (e.g., have different chain lengths or hydrophobicity). More preferably, the first protein amphiphile or bonded polypeptide and second protein amphiphile or bonded polypeptide are first dissolved or dispersed in an aqueous solvent to form respective aqueous solutions, which are then mixed together. In a preferred embodiment, the aqueous solvent is water with a salinity (i.e., salt concentration) from 0.1 μM to 10 M, more preferably from 10 μM to 2 M, and most preferably from 100 μM to 1 M. The concentration of each fusion protein or bonded polypeptide in their respective solutions will vary, but preferably ranges from about 0.01 μM to about 10 mM, more preferably from about 0.1 μM to about 1 mM, and even more preferably from about 0.1 μM to about 100 μM. In some embodiments where folded, globule proteins are hydrophilic headgroup and elastin-like peptides are hydrophobic tail, the fusion protein or bonded polypeptide containing the folded, globule proteins and the fusion protein or bonded polypeptide containing the elastin-like peptides are then preferably mixed at a molar ratio of from about 1:1000 to about 10:1, more preferably from about 1:100 to about 1:1, and most preferably from about 1:100 to 1:20. The concentration of the first fusion protein or bonded polypeptide in the combined solution preferably ranges from about 0.01 μM to about 10 mM, more preferably from about 0.1 μM to about 1 mM, and even more preferably from about 0.1 μM to about 100 μM. The total concentration of the mixture polypeptides in the solution will vary, but preferably ranges from about 0.1 μM to about 10 mM, more preferably from about 10 μM to about 1 mM, and even more preferably from about 30 μM to about 1 mM.

The vesicle formation solution is then allowed to stand under ambient conditions at room temperature (25° C.) or at a temperature above Tₜ with phase transition peptides for at least about 0.05 hours, more preferably from about 0.5 to about 3 hours, and even more preferably for about 1 hour. The formed protein vesicles have an average hydrodynamic diameter ranging from about 10 nm to about 1 mm, more preferably from about 100 nm to 100 μm, and even more preferably from 500 nm to 10 μm.

Alternatively, once the two or more pluralities of protein amphiphiles or bonded polypeptides are mixed, the solvent is then removed, preferably under vacuum, to produce a dried mixture. In one embodiment, the protein amphiphiles are lyophilized. The dried mixture preferably comprises less than about 10% by weight moisture, and more preferably less than about 5% by weight moisture, based upon the total weight of the dried mixture taken as 100% by weight. Once the solvent is removed, the dried protein or polypeptides mixture is then rehydrated with cold buffer and warmed to room temperature until the final desired concentration of each protein amphiphile dissolved in water is reached and vesicles are formed.

### IV. Methods of Using Protein Vesicles

A Targeting and Active Agent Delivery

In one embodiment, the protein amphiphiles incorporated as the building block of the vesicles or proteins bound to the surface of these vesicles can recognize a receptor expressed on the surface of cells of targeted tissue. These tissues include but are not limited to tumor tissue, vasculature, cardiac, nervous system, mucosal, dermal tissue, etc. In some preferred embodiment, the targeting globule protein domain facing outward on the membrane of the protein vesicles selectively bind to cells of targeted tissue, and the encapsulated active agents are delivered to the cells of targeted tissue.

The protein vesicles can be administered prophylactically, therapeutically, or combinations thereof. Therefore, the protein vesicles can be administered during a period before, during, or after onset of one or more symptoms of a disorder. In some embodiments, protein vesicle is administered with one or more additional therapeutic agents as part of a co-therapy, one or more second treatments, or combinations thereof. Second treatments include, but are not limited to, diet and exercise regimes, nutrition supplementation, vitamin supplementation, incontinence management, rehabilitative therapy, cognitive therapy, and management of inadvertent adverse effects or complications resulting from medical treatment.

The protein vesicles and the second therapeutic agent or treatment can be administered to the subject together or separately. The protein vesicles and the additional therapeutic agent or treatment can be administered on the same day, on a different days, or combinations thereof.

For example, the subject can be administered the disclosed composition 0, 1, 2, 3, 4, 5, or more days before administration of or exposure to the additional therapeutic agent or treatment. In some embodiments, the subject can be administered one or more doses of the composition every 1, 2, 3, 4, 5, 6 7, 14, 21, 28, 35, or 48 days prior to a first administration of or exposure to the additional therapeutic agent or treatment.

The subject can also be administered the composition for 0, 1, 2, 3, 4, 5, or more days after administration of or exposure to the additional therapeutic agent or treatment. The subject can also be administered the composition during administration of or exposure to the additional therapeutic agent or treatment. The composition can be administered on the same day as the antineoplastic agent, or on a different day. The subject can be administered one or more doses of the composition every 1, 2, 3, 4, 5, 6, 7, 14, 21, 28, 35, or 48 days during or after administration of the additional therapeutic agent or treatment.

Preferred methods of administration include systemic or local administration. The protein vesicles can be combined in admixture with a pharmaceutically acceptable carrier vehicle. Therapeutic formulations are prepared for storage in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween®, Pluronics® or PEG.

The compositions of the present disclosure can be administered parenterally. As used herein, “parenteral administration” is characterized by administering a pharmaceutical composition through a physical breach of a subject's tissue. Parenteral administration includes administering by injection, through a surgical incision, or through a tissue-penetrating non-surgical wound, and the like. In particular, parenteral administration includes subcutaneous, intraperitoneal, intravenous, intraarterial, intramuscular, intrasternal injection, and kidney dialytic infusion techniques.

Parenteral formulations can include the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Parenteral administration formulations include suspensions, solutions, emulsions in oily or aqueous vehicles, pastes, reconsitutable dry (i.e. powder or granular) formulations, and implantable sustained-release or biodegradable formulations. Such formulations may also include one or more additional ingredients including suspending, stabilizing, or dispersing agents. Parenteral formulations may be prepared, packaged, or sold in the form of a sterile injectable aqueous or oily suspension or solution. Parenteral formulations may also include dispersing agents, wetting agents, or suspending agents described herein. Methods for preparing these types of formulations are known. Sterile injectable formulations may be prepared using non-toxic parenterally-acceptable diluents or solvents, such as water, 1,3-butane diol, Ringer's solution, isotonic sodium chloride solution, and fixed oils such as synthetic monoglycerides or diglycerides. Other parentally-administrable formulations include microcrystalline forms, liposomal preparations, and biodegradable polymer systems. Compositions for sustained release or implantation may include pharmaceutically acceptable polymeric or hydrophobic materials such as emulsions, ion exchange resins, sparingly soluble polymers, and sparingly soluble salts.

Pharmaceutical compositions may be prepared, packaged, or sold in a buccal formulation. Such formulations may be in the form of tablets, powders, aerosols, atomized solutions, suspensions, or lozenges made using known methods, and may contain from about 0.1% to about 20% (w/w) active ingredient with the balance of the formulation containing an orally dissolvable or degradable composition and/or one or more additional ingredients as described herein. Preferably, powdered or aerosolized formulations have an average particle or droplet size ranging from about 0.1 nanometers to about 200 nanometers when dispersed.

The composition can include one or more additional ingredients. As used herein, “additional ingredients” include: excipients, surface active agents, dispersing agents, inert diluents, granulating agents, disintegrating agents, binding agents, lubricating agents, sweetening agents, flavoring agents, coloring agents, preservatives, physiologically degradable compositions (e.g., gelatin), aqueous vehicles, aqueous solvents, oily vehicles and oily solvents, suspending agents, dispersing agents, wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other additional ingredients which may be included in the pharmaceutical compositions are known. Suitable additional ingredients are described in Remington's Pharmaceutical Sciences, 17^{th} ed. Mack Publishing Co., Genaro, ed., Easton, Pa. (1985).

B. Dosages

Dosages and desired concentrations of the polynucleotide-binding polypeptide disclosed herein in pharmaceutical compositions of the present disclosure may vary depending on the particular use envisioned. The determination of the appropriate dosage or route of administration is well within the skill of an ordinary physician Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

The composition can be administered intravenously in a wide dosing range from about 0.01 milligram per kilo body weight (mg/kg) to about 1.0 mg/kg, depending on patient's age and physical state, as well as dosing regimen and schedule.

C Packing Material

1. Column Chromatography

In some embodiments, the protein vesicles are packed onto a column as the solid medium for separating biochemical mixtures in chromatography. In further embodiments, the protein vesicles are used as the solid medium in affinity chromatography based on its capability to permit specific interactions between antigen and antibody, enzyme and substrate, or receptor and ligand.

In a preferred embodiment, folded proteins of specific antigens, enzymes, or receptors, or fragments thereof, are used as the hydrophilic headgroup in the formation of self-assembled vesicles. The formed vesicles with antigens, enzymes, or receptors facing outside vesicles are packed onto a column; subsequently, the initial mixture is run through the column to allow binding to the protein vesicles via highly specific interactions with the proteins on the membrane surface of the vesicles; a wash buffer is later applied through the column to remove un-bound sample; finally, the elution buffer is applied to the column and the target molecule is collected. Alternatively, binding may be achieved using a batch treatment, for example, by adding the initial mixture to the protein vesicles in a vessel, mixing, separating the protein vesicles, removing the liquid phase, washing, re-centrifuging, adding the elution buffer, re-centrifuging and removing the eluate. Sometimes a hybrid method is employed such that the binding is done by the batch method, but the protein vesicle solid phase with the target molecule bound is packed onto a column and washing and elution are done on the column. A third method, expanded bed adsorption, which combines the advantages of the two methods mentioned above, has also been developed. The solid phase protein vesicles are placed in a column where liquid phase is pumped in from the bottom and exits at the top. The gravity of the protein vesicles ensure that the solid phase does not exit the column with the liquid phase.

2. Template/Casting

In some embodiments, the protein vesicles are dispersed in an appropriate solvent (generally water) to serve as template particles to ultimately fabricate porous materials. In further embodiments, the protein vesicles are used in freeze-casting where ice crystals nucleate and redistribute the vesicles when subjecting the aqueous slurry of protein vesicles to a directional temperature gradient. Once solidification has ended, the frozen, templated protein vesicles is placed into a freeze-dryer to remove the ice crystals. The resulting body contains macropores in an exact replica of the sublimated ice crystals and micropores found between the vesicles in the walls.

In an alternative embodiment, the protein vesicles are added to a solution of dissolved polymer and the mixture is shaped into its final geometry, e.g. cast on a surface to produce a membrane or in a three-dimensional mold to produce a scaffold. When the solvent evaporates, a structure of composite material containing the vesicles together with the polymer is formed. Upon temperature change in the embodiments of protein vesicles formed with elastin-like peptides, or upon submerging in a bath which dissembles the protein vesicles, the composite material leaves behind a porous structure.

D. Micro-/Nano-Reactors

In some embodiments, the protein vesicles are used as micro-sized or nano-sized reactors. Chemical or biological reactions can be carried out in the non-continuous phase of non-polar or low-polarity environment near the interior wall of the protein vesicles. Chemical or biological reactions can also be carried out in the aqueous phase.

In some embodiments, the interior hollow space of the vesicles permits chemical or biological reactions. In some further embodiments, the interior hollow space of the vesicles can be further compartmentalized (e.g. by nanospheres) to permits multi-step reactions.

E. Surfactants

1. Cleaning Detergents

It is contemplated that the protein vesicles containing enzymes or other catalysts of the present invention will find use in any suitable cleaning composition, including but not limited to bar and liquid soap applications, dishcare formulations, surface cleaning applications, contact lens cleaning solutions or products, waste treatment, textile applications, pulp-bleaching, disinfectants, skin care, oral care, hair care, etc.

In some embodiments, protein vesicles contain enzymes that break down proteins, starches, fats and grease and can be used as washing powder. These enzymes can be the hydrophilic headgroup facing outward on the vesicle membrane. In some further embodiments, elastin-like peptides of the protein vesicles can be solubilized to release a second plurality of enzymes or other active agents that are encapsulated in the vesicles, when the temperature decreases from above Tₜ to below Tₜ, as in a warm-to-cold washing cycle.

2. Cosmetics

In some embodiments, the protein vesicles are used as surfactants in the cleansing, emulsification, solubilization, and conditioning applications. In further embodiments, the protein vesicles help suspend oil and remove oily debris in a cosmetic application. Formulations that contain active agents, e.g. vitamins, salts, peptides, proteins, and polymers with hydrating effects, are applied in these protein vesicles.

### EXAMPLES

### Example 1

### Preparation of Recombinant Proteins and Thermally Triggered Self-Assembly of Protein Vesicles

### Materials and Methods

Materials

Oligonucleotide primers were synthesized by Eurofins MWG Operon, and DNA polymerase (PfuUltra II Fusion HS) was purchased from Agilent Technologies. E. coli strains XL1-Blue (Agilent Technologies), AFIQ-BL21 (Datta D, et al., J Am. Chem. Soc., 124 (20), 5652-5653 (2002)), and the plasmids containing Z_{R}-ELP, mCherry, and Z_{E} fragments (Zhang K, et al., J. Am. Chem. Soc, 127, 10136-10137 (2005)) were obtained accordingly. The expression vector pQE60 (3.4 kbp) and nickel nitrilotriacetic acid (Ni-NTA) resin were purchased from Qiagen.

Plasmid Construction

Plasmids for EGFP-Z_{E} (pQE60-EGFP-Z_{E}) were constructed by assembly PCR technique that combined the two gene fragments. Following the same procedure described in our previous work to construct the plasmid pQE60-mCherry-Z_{E} (Park W M, et al., Angew. Chem. Int. Ed., 52 (31), 8098-8101 (2013)), the gene encoding EGFP-Z_{E} was constructed by two-step PCR. In the first step, two gene fragments encoding either EGFP or Z_{E} were amplified using two pairs of primers E1 (5′-TATCATAGATCTATGGCTAGCAAAGGAGAAGAACTCTTC-3′) (SEQ ID NO:22) and E2 (5′-CAGGGATCCACTACCGCGAAGATTGTACAGTTCATCCATGCCATGTG TAATC-3′) (SEQ ID NO:23) and primers Z1 (5′-CTTCGCGGTAGTGGATCCCTGGAAATCGAAGC-3′) (SEQ ID NO:24) and Z2 (5′-TATCATAGATCTCAGCGGACCGTAACGGGTAC-3′) (SEQ ID NO:25), respectively. In the second step, the amplified gene fragments were assembled using primers E1 and Z2. The resulting DNA construct was digested and ligated into the BglII restriction site of the pQE60 vector. Both mCherry-Z_{E} and EGFP-Z_{E} were tagged with a Hiss tag to their C-termini for purification.

Protein Expression

Z_{R}-ELP was co-expressed with His₆-tagged Z_{E}, e.g. a peptide having an amino acid sequence of

| (SEQ ID NO: 26) | |
|---|---|
| LEIEAAALEQ ENTALETEVA ELEQEVQRLE NIVSQYRTRY GPLRSHHHHH H | 51 |

to prevent degradation during expression (Park W M, et al., Angew. Chem. Int. Ed., 52 (31), 8098-8101 (2013); Zhang K, et., Chem Bio Chem, 10 (16), 2617-2619 (2009)). Co-expressed Z_{R}-ELP, i.e. the fusion peptide having an amino acid sequence of MKGSLEIRAAALRRRNTALRTRVAELRQRVQRLRNEVSQYETRYGPL(G₄S)₂G[(VPGVG)₂VPGFG(VPGVG)₂]₅VPGC (SEQ ID NO:27) and His₆Z_{E} form a protein complex His₆Z_{E}/Z_{R}-ELP, which is resistant to proteolysis in the cytoplasm. All plasmids (pQE60-His₆Z_{E}/Z_{R}-ELP, pQE60-mCherry-Z_{E}, and pQE60-EGFP-Z₅) were transformed into E. coli strain AFIQ-BL21 for expression. All cell cultures were grown at 37° C. in 2× yeast extract and tryptone (YT) media containing ampicillin (200 mg/L) and chloramphenicol (34 mg/L). When the cell culture optical density at 600 nm reached 0.8, protein expression was induced by 1.0 mM of isopropylβ-thiogalactoside (IPTG). Cells were harvested by centrifugation after 5 hours of expression at 37° C.

Protein Purification and Analysis

Z_{R}-ELP: Purification of Z_{R}-ELP was conducted as previously reported (Park W M, et al., Angew. Chem. Int. Ed., 52 (31), 8098-8101 (2013); Zhang K, et., Chem Bio Chem, 10 (16), 2617-2619 (2009)). Briefly, the crude lysate containing complexes of the co-expressed His₆Z_{E} and Z_{R}-ELP was produced from harvested cells under denaturing conditions, and incubated with Ni-NTA resin. After washing, Z_{R}-ELP was collected from elution buffer containing 6 M guadinine hydrochloride that isolated Z_{R}-ELP from His₆Z_{E} on the NTA resin. Purified proteins were dialyzed into deionized water.

mCherry-Z_{E}: Expressed mCherry-Z_{E} having an amino acid sequence of

| (SEQ ID NO: 28) | |
|---|---|
| MGGSRSMVSK GEEDNMAIIK EFMRFKVHME GSVNGHEFEI EGEGEGRPYE GTQTAKLKVT | 60 |
| KGGPLPFAWD ILSPQFMYGS KAYVKHPADI PDYLKLSFPE GFKWERVMNF EDGGVVTVTQ | 120 |
| DSSLQDGEFI YKVKLRGTNF PSDGPVMQKK TMGWEASSER MYPEDGALKG EIKQRLKLKD | 180 |
| GGHYDAEVKT TYKAKKPVQL PGAYNVNIKL DITSHNEDYT IVEQYERAEG RHSTGGMDEL | 240 |
| YKSKLRGSGS LEIEAAALEQ ENTALETEVA ELEQEVQRLE NIVSQYRTRY GPLRSHHHHH | 300 |
| H | 301 |

was purified following previously reported procedure (Park W M, et al., Angew. Chem. Int. Ed., 52 (31), 8098-8101 (2013)). All buffers contained 8 M urea, 10 mM Tris-Cl, and 100 mM Na₂HPO₄ and were adjusted to different pH values. Harvested cells were frozen, thawed, and sonicated in buffer at pH 8.0, and the cell lysate was cleared by centrifugation. The cleared cell lysate was incubated with Ni-NTA resin. After washing at pH 6.3, the protein was eluted from the resin at pH 4.5. After dialysis into phosphate buffered saline (PBS, pH 7.4), aggregates of insoluble protein were removed by centrifugation.

EGFP-Z_{E}: EGFP-Z_{E} having an amino acid sequence of

| (SEQ ID NO: 29) | |
|---|---|
| MGGSRSMASK GEELFTGVVP ILVELDGDVN GHKFSVSGEG EGDATYGKLT LKFICTTGKL | 60 |
| PVPWPTLVTT LCYGVQCFSR YPDHMKRHDF FKSAMPEGYV QERTIFFKDD GNYKTRAEVK | 120 |
| FEGDTLVNRI ELKGIDFKED GNILGHKLEY NYNSHNVYIM ADKQKNGIKV NFKTRHNIED | 180 |
| GSVQLADHYQ QNTPIGDGPV LLPDNHYLST QSALSKDPNE KRDHMVLLEF VTAAGITHGM | 240 |
| DELYNLRGSG SLEIEAAALE QENTALETEV AELEQEVQRL ENIVSQYRTR YGPLRSHHHH | 300 |
| HH | 302 |

was purified using Ni-NTA under native conditions. All prepared buffers contained 50 mM Na₂HPO₄, 300 mM NaCl, and 10-250 mM imidazole at pH 8.0. After cell lysis by freeze-thaw and sonication, the cleared cell lysate in buffer containing 10 mM imidazole was incubated with Ni-NTA, washed (20 mM imidazole), and eluted in the presence of 250 mM imidazole. The eluted solution was dialyzed into PBS (pH 7.4).

Analysis:

Purified proteins were analyzed using tricine sodium dodecyl sulfate polyacrylamide gel electrophoresis (SDS-PAGE). In order to avoid hydrolysis during boiling samples (Park W M, et al., Angew. Chem. Int. Ed, 52 (31), 8098-8101 (2013); Gross L A, et al., Proc. Natl. Acad. Sci. U.S.A, 97 (22), 11990-11995 (2009)), all protein samples were prepared by reducing proteins at room temperature for 30 min.

Self-Assembly of Vesicles

Solutions of the purified recombinant proteins, Z_{R}-ELP, mCherry-Z_{E}, and EGFP-Z_{E} were mixed on ice. The final protein and salt concentration was adjusted by adding deionized water and diluting concentrated buffer (100 mM Na₂HPO₄, 18 mM KH₂PO₄, 27 mM KCl, and 1.37 M NaCl, pH 7.4). The protein mixture solutions were left on ice for 15 min. Next, the solutions were placed at room temperature (25° C.) for 1 hour to form vesicles. For imaging purposes, a protein solution (300 μl) containing mCherry-Z_{E} (1.5 μM) and Z_{R}-ELP (30 μM) was prepared in a cuvette at 4° C. with a salt concentration of 0.3 M, and the photographs were taken at 0, 5, 15, 30, 60 min after placing at room temperature (25° C.).

Confocal Microscopy

Confocal micrographs were obtained with a Zeiss LSM 510Vis (Carl Zeiss). A droplet of vesicle samples (5 μl) was placed on a rectangular glass (24×60 mm), which was covered with a square cover glass (18×18 mm) in the presence of spacers with thickness of 0.15 mm. All vesicle samples were imaged using a 100× oil immersion objective. Laser wavelengths of 488 nm and 543 nm were used with high pass (LP 505) and band pass (BP 565-615) emission filters.

Scanning Electron Microscopy (SEM)

A cross-section of freeze-dried fractured vesicles was imaged with a Zeiss Ultra60 FE-SEM (Carl Zeiss). A vesicle sample was placed on a glass substrate and fixed with glutaraldehyde (1.0%) for 1 hour. After washing twice with deionized water, the samples were freeze-dried and fractured. The vesicles were transferred to a carbon tape and sputter-coated with gold before imaging at 5 kV. Non-fractured vesicles were also imaged as a control. The thickness of vesicle membrane was measured from a close-up image of a fractured vesicle.

Dynamic Light Scattering (DLS)

Hydrodynamic diameters of vesicles were measured with a Zetasizer Nano ZS (Malvern Instrument). A 4 mW He—Ne laser operating at a wavelength of 633 nm was equipped and operated at a detection angle of 173°. Vesicle solution samples (100 μl) were prepared in a cuvette, and measurements were performed at 25° C. The raw correlation data were processed to size distribution by using Dispersion Technology Software.

Circular Dichoism (CD) Spectroscopy

The CD spectra of protein complexes were recorded on a Jasco J-810 spectropolarimeter (JASCO). Protein solution samples were prepared in PBS (pH 7.4), and measurements were performed in a 0.2 cm-length cuvette. The spectra were obtained in 1 nm increments within a wavelength range of 200-300 nm. Mean residue ellipticity was measured from protein solutions containing 5 μM of protein complexes at 4° C. Mean residue ellipticity was converted from accumulated spectra of five measurements for each sample. The peak at a wavelength of 222 nm indicated presence of α-helical coiled-coils of Z_{R}/Z_{R} and Z_{E}/Z_{R}.

### Results

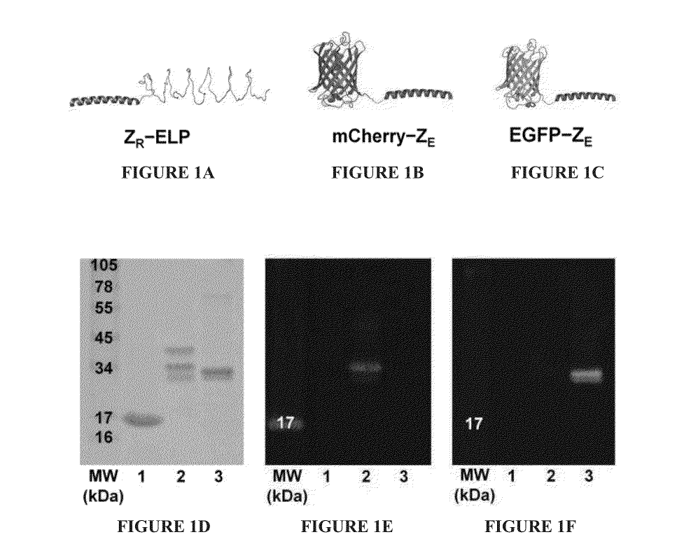

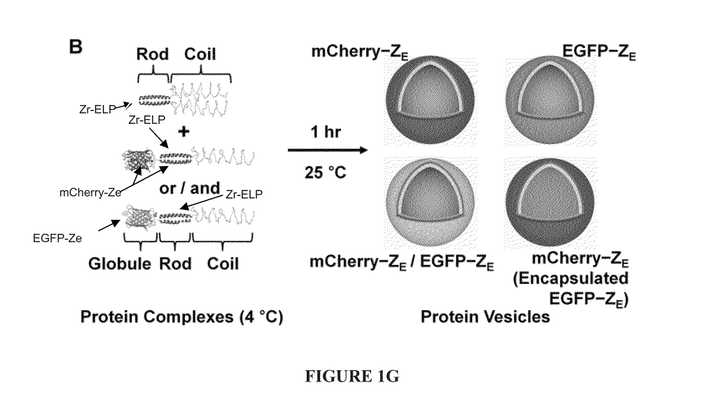

Protein vesicles were created by combinations of three different diblock recombinant proteins: (i) an arginine-rich leucine zipper motif (Z_{R}) fused with an ELP (FIG. 1A),i.e., Z_{R}-ELP (Zhang K, et al., Chem Bio Chem, 10, 2617 (2009)); (ii) a fluorescent, globular protein, mCherry, fused with a glutamic acid-rich leucine zipper motif (Z_{E}), i.e., mCherry-Z_{E} (FIG. 1B); and (iii) an enhanced green fluorescent protein (EGFP) fused with a Z_{E}, i.e., EGFP-Z_{E} (FIG. 1C). SDS-PAGE shows that Z_{R}-ELP, mCherry-Z_{E}, and EGFP-Z_{E} were purified (FIG. 1D). Since mCherry-Z_{E} and EGFP-Z_{E} are resistant to denaturation by SDS, fully and partially folded fractions appeared as different bands in the gels. Fluorescence from those bands was imaged using a fluorescence/phospho-imager (Typhoon, GE Healthcare Life Sciences) to confirm that they are from either mCherry-Z_{E} or EGFP-Z_{E}, not impurities (FIG. 1E, FIG. 1F).

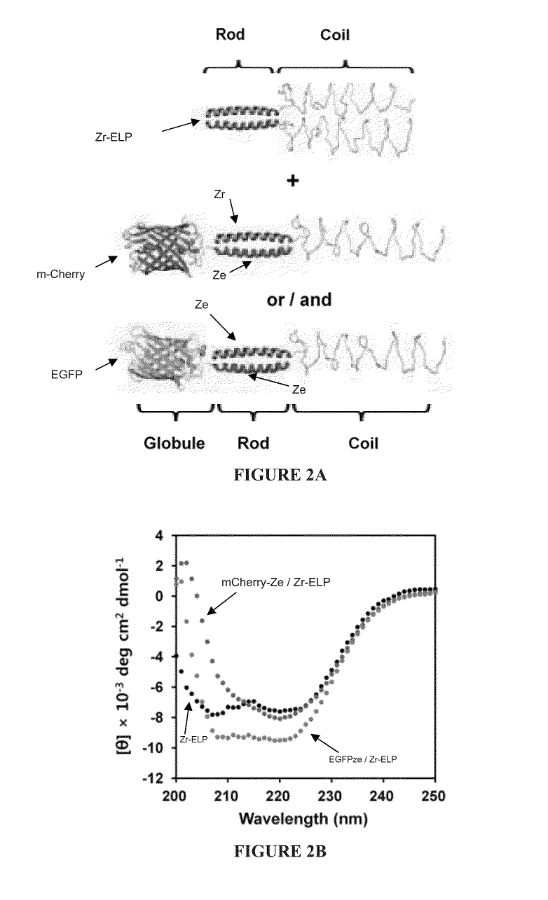

The Z_{R} forms coiled coil complexes with its counterpart Z_{E} at a high affinity, whose dissociation constant is extremely low (K_{d}≈10⁻¹⁵ M) (Moll J R, et al., Protein Sci., 10, 649 (2001)), forming the hydrophilic “rod” structure of Z_{E}/Z_{R}. The Z_{R} coil has a weaker affinity with another Z_{R} motif (K_{d}≈10⁻⁷ M) (Moll J R, et al., Protein Sci., 10, 649 (2001)). By mixing the protein solutions at 4° C., mCherry-Z_{E} or EGFP-Z_{E} were incorporated with Z_{R}-ELP, forming a “globule-rod-coil” amphiphilic protein complex (mCherry-Z_{E}/Z_{R}-ELP, or EGFP-Z_{E}/Z_{R}-ELP), whereas Z_{R}-ELP formed homodimers in a “rod-coil” amphiphilic protein complex (FIG. 2A). Circular dichroism spectroscopy confirmed that each protein complex contained the α-helical coiled coil motifs (FIG. 2B).

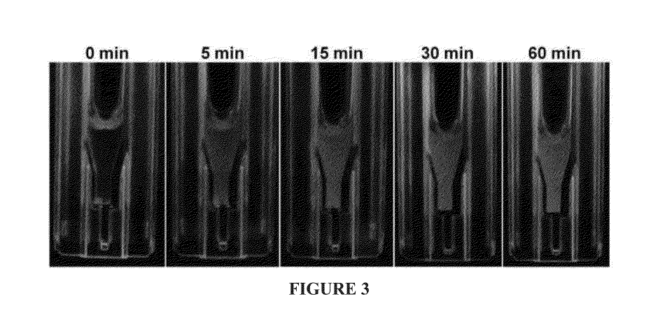

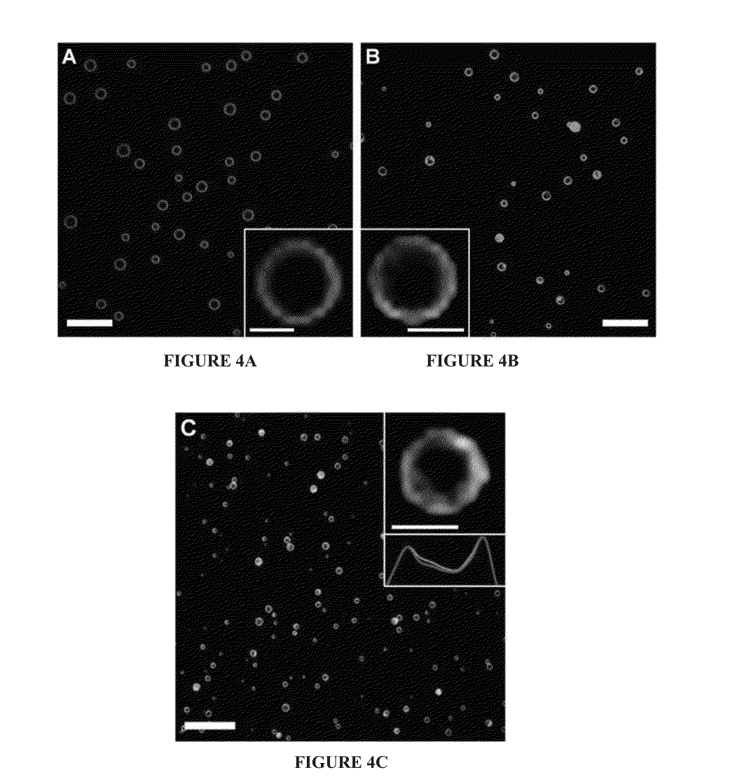

The protein mixture solution was placed at room temperature for an hour, and the ELP domain underwent an inverse phase transition into a hydrophobic phase, facilitating the formation of protein vesicles. The mixture solution became more and more turbid from 0, 5, 15, 30, and 60 minutes after placed at room temperature as a result of the phase transition and vesicle formation (FIG. 3). Protein vesicles self-assembled from Z_{R}-ELP mixed with either mCherry-Z_{E} or EGFP-Z_{E} were visualized under fluorescent microscopy, showing a round, hollow structure whose membrane was either red or green due to the presence of the fluorescent protein domain (FIG. 4A, FIG. 4B). 1.5 μM of mCherry-Z_{E} and 30 μM of Z_{R}-ELP in an aqueous solution of a salt concentration of 0.30 M formed vesicles with an average diameter of 1.26 μm and a narrow size distribution (polydispersity index<0.03), according to dynamic light scattering (DLS) measurements. 0.6 μM of EGFP-Z_{E} and 30 μM of Z_{R}-ELP in an aqueous solution of a salt concentration of 0.91 M formed vesicles with an average diameter of 1.82 μm and a narrow size distribution (polydispersity index<0.03). The red and green fluorescence indicated homogeneous incorporation of mCherry-Z_{E} and EGFP-Z_{E}, in each vesicle membrane. When both mCherry-Z_{E} and EGFP-Z_{E} are mixed with Z_{R}-ELP at 4° C. at 0.3 μM, 0.3 μM and 30 μM, respectively, with a salt concentration of 0.91 M, they self-assembled at room temperature into hollow vesicles incorporating both globular domains in the membrane. Fluorescent confocal microscopy showed that red (mCherry-Z_{E}) and green (EGFP-Z_{E}) colocalized to yellow on the membrane surface of the hollow vesicles (FIG. 4C); the fluorescence intensity profile analysis confirmed the colocalized red and green signal at the membrane of the vesicles (FIG. 4C).

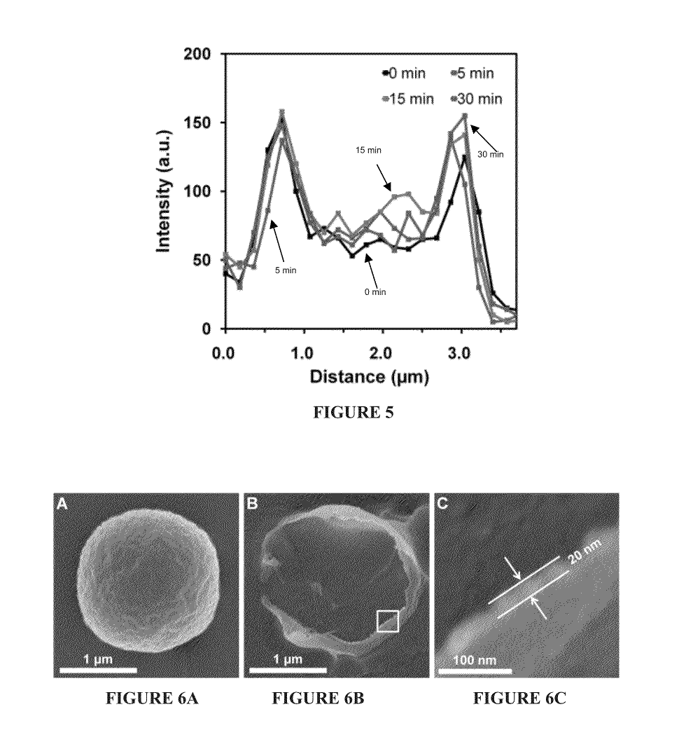

No loss of fluorescence by denaturation of mCherry or EGFP was seen, as the inverse phase transition of ELF did not involve the use of any organic solvents and thus in a biocompatible environment. Upon dilution of the vesicle solution, no significant change in the fluorescence intensity was observed (FIG. 5), indicating that mCherry-Z_{E} is not exchanged between vesicles and solution due to the extremely low dissociation constant of Z_{E} and Z_{R} coiled coils. To confirm that the vesicles were hollow, cross-sections of fractured, freeze-dried vesicles were imaged under scanning electron microscopy (SEM), which showed the empty inner space of a vesicle (FIG. 6A, FIG. 6B), The thickness of the vesicle membrane was about 20 nm, as measured from SEM images (FIG. 6C).

### Example 2

### Salt Concentration Determines the Formation of Vesicle

### Materials and Methods

Turbidity Measurement

The turbidity of protein solutions was measured from the optical density of transmitted light at 400 nm, using a microplate reader (Synergy HT Multi-Mode, BioTeck). Absorption by the proteins is negligible at this wavelength. The protein solutions (100 μl) were prepared in a 96-well microplate at 4° C. and placed in the instrument at 25° C. Then, the changes of turbidity were monitored by recording the optical density of protein solutions every minute for 100 min.

### Results

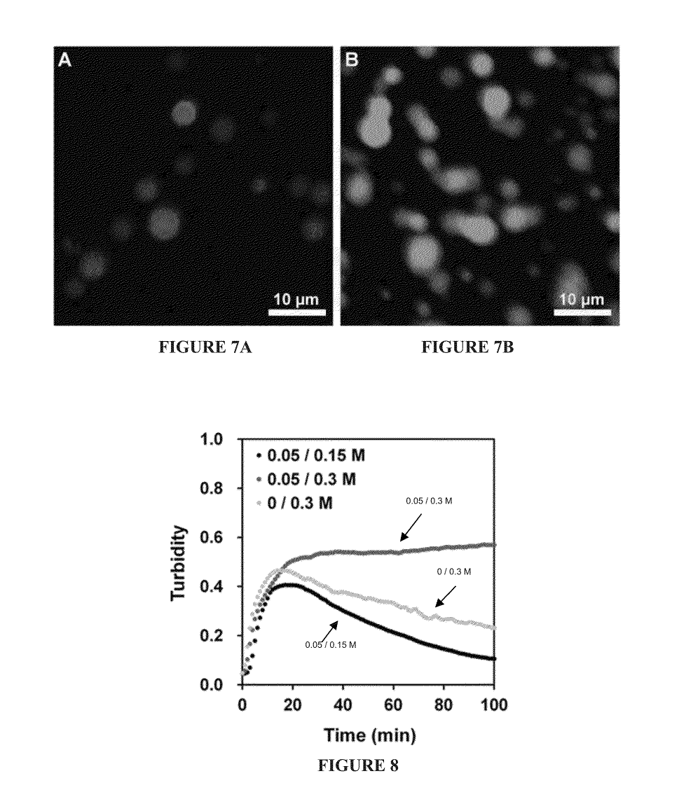

The inverse phase transition of protein mixture solutions were tested at different salt concentrations (0.15 M-1.21 M) with a fixed concentration of Z_{R}-ELP (30 μM). Vesicle formation was only observed above critical values of salt concentration, which are estimated to be approximately 0.30 and 0.91 M for vesicles incorporating mCherry-Z_{E} and EGFP-Z_{E}, respectively. Below these concentrations, only the formation of coacervate particles was observed, which were droplets of protein-rich phases (FIG. 7A, FIG. 7B). They were the typical result of ELF inverse phase transition (Cirulis J T, et al., Biochemistry, 49, 5726 (2010); Osborne J L, et al., Acta Biomater., 4, 49 (2008)).

As probed by turbidity profiles, molecular packing of vesicles was distinct from that of coacervate particles (FIG. 8). After an initial rapid increase, saturation of turbidity was observed at the salt concentrations for vesicle formation, indicating the surface of vesicles was hydrophilic and stable. In contrast, there was a slow decrease in turbidity when formation of coacervate particles was favored, either at lower salt concentrations or in the absence mCherry-Z_{E} or EGFP-Z_{E}. The decrease in turbidity is caused by coalescence of protein coacervate particles (Cirulis J T, et al., Biochemistry, 49, 5726 (2010)), which indicates that they have hydrophobic surfaces where ELP motifs are exposed to water.

The effect of salt concentration can be further rationalized using the packing parameter (Israelachvili J N, Intermolecular and Surface Forces: Revised Third ed., Elsevier Science: Waltham, Mass. (2011)):

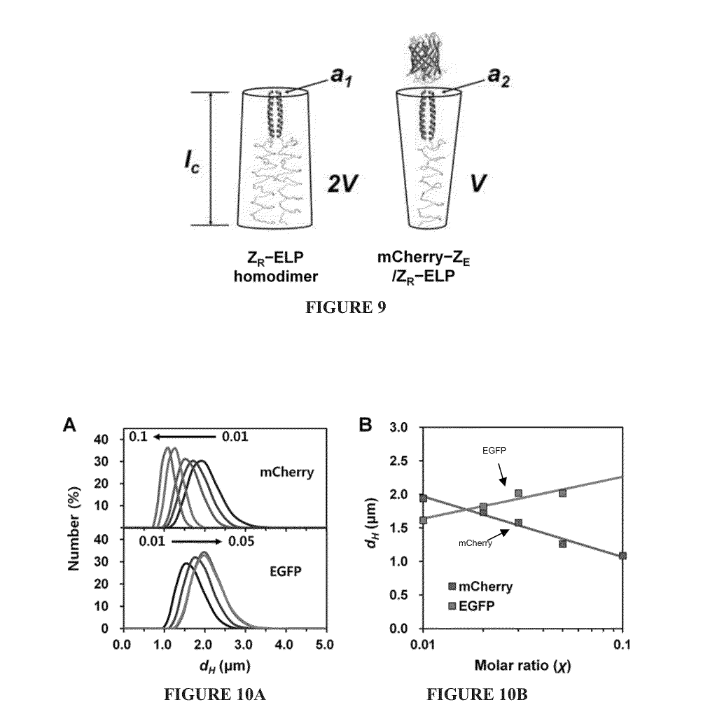

P=V/(a₀l_{c})

where V is the volume of the hydrophobic (ELP) block, a₀ is the average head area of the hydrophilic block, and l_{c} is the critical length (FIG. 9). The hydrophilic part is composed of the globular domain (mCherry or EGFP) and the rod-shaped coiled coils (mixtures of Z_{R} Z_{R} homodimers or Z_{E}/Z_{R} heterodimers). When mCherry-Z_{E} (or EGFP-Z_{E}) is mixed with Z_{R}-ELP, the average head area per single strand of ELP (a₀) is expressed as:

a₀=(1−χ)a₁/2+χa₂

where χ is the molar ratio of mCherry-Z_{E} (or EGFP-Z_{E}) to Z_{R}-ELP, and a₁ and a₂ are the head areas of Z_{R} Z_{R} and mCherry (or EGFP)-Z_{E}/Z_{R}, respectively (FIG. 9).

Because of fixed secondary structure and surface properties of the globule and rod blocks, a₀ should not strongly depend on salt concentration. Conversely, V is significantly influenced by ionic strength. According to conformational mechanics of ELPs (Valiaev A, et al., J. Am. Chem. Soc., 130, 10939 (2008)), ELP molecules are more collapsed with increasing salt concentration. Therefore, increased ionic strength reduces V and decreases the packing parameter P.

Hence, the protein amphiphiles have an inverted cone shape that forms coacervate particles when P>1, below the critical values of salt concentration. Above the critical values, V is reduced, and vesicle formation is favored at ½<P<1. Therefore, conformational dependency of ELF on ionic strength seems strongly related to morphologies of the aggregates made from the protein amphiphiles.

The critical salt concentration is dependent on the type of globular domains. The head area a₂ is decided by the nature of globular domains, as they are fused, i.e.; covalently bonded, with Z_{E} Z_{R} coiled coils (FIG. 9). For example, mCherry is a monomeric and highly soluble globular protein (Lam C N, et al., Biomacromolecules, 15, 1248 (2014)), and a₂ becomes larger than the head area resulted only from a coiled coil (˜a₁) due to steric hindrance provided by the bonded mCherry domain. In contrast, EGFP tends to dimerize at millimolar concentration (Zacharias D A, et al., Science, 296, 913 (2002)) or even can aggregate as indicated by the bright spots in the fluorescent images of EGFP-Z_{E}/Z_{R}-ELP vesicles (FIG. 4B). The attraction between EGFP domains reduces a₂ corresponding to EGFP-Z_{E}/Z_{R} block. Thus, the salt concentration required for ½<P<I should be higher for EGFP than mCherry.

### Example 3

### Diameter of Vesicles Varies Differently with Different

Globular Proteins

### Materials and Methods

Self-assembly of vesicles was performed following the same procedure as described in Example 1.

### Results

As the molar ratio (χ) of mCherry-Z_{E} (or EGFP-Z_{E}) to Z_{R}-ELP increased at a given salt concentration, the average hydrodiameter (d_{H}) of mCherry-Z_{E}/Z_{R}-ELP vesicles decreased, whereas the d_{H} of EGFP-Z_{E}/Z_{R}-ELP vesicles increased (FIG. 10A, FIG. 10B).

The increased curvature of mCherry-Z_{E}/Z_{R}-ELP vesicles at higher χ was believed to indicate a₂>a₁, whereas the decreased curvature of EGFP-Z_{E}/Z_{R}-ELP vesicles at higher χ was believed to indicate a₂<a₁. As defined in Example 2, a₁ is the head area of the “rod” structure, Z_{R} Z_{R}, and a₂ is the head area of the “globule-rod” structure, mCherry (or EGFP)-Z_{E}/Z_{R}. Hence, mCherry was believed to provide a larger head area than EGFP. The opposite trends of d_{H} as both mCherry-Z_{E} and EGFP-Z_{E} were equally increased compared to Z_{R}-ELP, were believed to indicate that the influence from the globular domains was dominant over interactions between Z_{R}/Z_{R} and Z_{E}/Z_{R} coiled coil domains. Nonetheless, various interactions between the protein domains may exist and contribute to self-assembly.

### Example 4

### Encapsulation of Protein Coacervates by Protein Vesicles

### Materials and Methods

Encapsulation of protein coacervates was performed by adding the Z_{E}-fused recombinant protein in the protein mixture solution in the beginning of the procedure as described in Example 1. The self-assembly process was performed accordingly.

### Results

At a salt concentration of 0.45 M with 30 μM of Z_{R}-ELP, only mCherry-Z_{E}/Z_{R}-ELP vesicles were favored and formed from a mixture of 1.5 μM mCherry-Z_{E}, 0.6 μM EGFP-Z_{E}, and 30 μM of Z_{R}-ELP. No EGFP-Z_{E}/Z_{R}-ELP vesicles were formed. Fluorescent confocal microscopy showed that mCherry-Z_{E} and Z_{R}-ELP self-assembled into vesicles whose interior was filled with EGFP-Z_{E} and Z_{R}-ELP (FIG. 11A, FIG. 11B). The two globular domains (mCherry and EGFP) separated into different microphases within a vesicle. The vesicular layer composed of mCherry-Z_{E} and Z_{R}-ELP encapsulated the coacervate phase of EGFP-Z_{E} and Z_{R}-ELP, which was simultaneously formed during the inverse phase transition. This condition (salt concentration of 0.45 M) resulted in vesicles with “core-shell” morphology, which was different from the hollow vesicles incorporating both globular domains in the membrane resulting from a salt concentration of 0.91 M (FIG. 4C), indicating the morphological dependence on salt concentration.

As probed by turbidity profiles, the saturation in turbidity after phase transition was believed to indicate that the “shell” is a stable vesicular membrane of tightly packed protein amphiphiles (FIG. 11C). At the same salt concentration, however, the turbidity profile for the mixture of only EGFP-Z_{E} and Z_{R}-ELP showed a gradual decrease, which was believed to indicate the formation of typical coacervate particles. This result demonstrates that vesicles incorporating multiple types of globular domains in either the membrane or interior compartment can self-assemble by adjusting salt concentration.

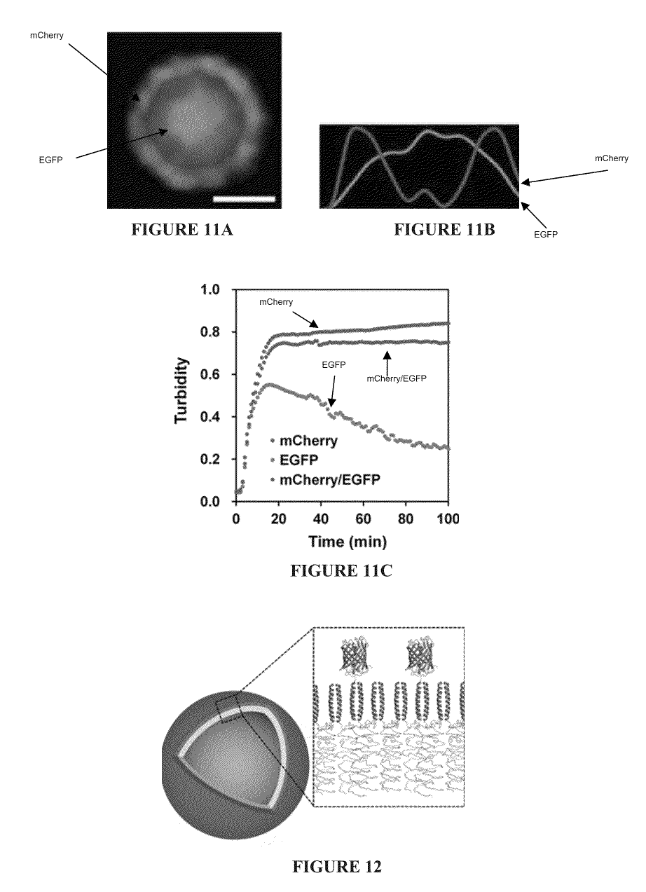

These vesicles were hypothesized to be composed of a self-assembled “single-layer” membrane, which could explain the preferential encapsulation of the coacervate phase. They were believed to be distinguished from the “bilayer” membranes of typical block copolymer vesicles. In a bilayer, hydrophilic chains are both inside and outside of vesicles. It was proposed here that the hydrophobic ELP blocks faced the interior (FIG. 12), and the inner surface could stabilize the encapsulated protein coacervate phase. An example of synthetic rod-coil block copolymers demonstrates that they form hollow aggregates where a hydrophobic inner shell encapsulates hydrophobic cargo (Jenekhe S A, et al., Science, 279, 1903 (1998)). Despite the globular domains included in the present system of mCherry-Z_{E} and EGFP-Z_{E}, this example shares the same characteristic of rod shaped blocks directly interfaced with hydrophobic coil blocks. It was hypothesized that the rigid, rod-shaped conformations could maintain a low interfacial curvature between the coiled coils and ELP (Bellomo E G, et al., Nat. Mater., 3, 244 (2004)) and may prevent the collapse of hollow structure even in the absence of encapsulated coacervate phase. Moreover, the observed correlation between packing parameter and curvature of vesicles (FIG. 10A, FIG. 10B) was similar to a characteristic of single-layer superstructures assembled from mesoscopic metal-polymer amphiphiles (Park S, et al., Science, 303, 348 (2004)).

### Example 5

### Encapsulation of Small Molecules by Protein Vesicles

### Materials and Methods

Encapsulation was performed following the same procedure as vesicle self-assembly, except that 50 μg/ml of fluorescein was added to protein mixture solutions on ice before room temperature incubation.

### Results

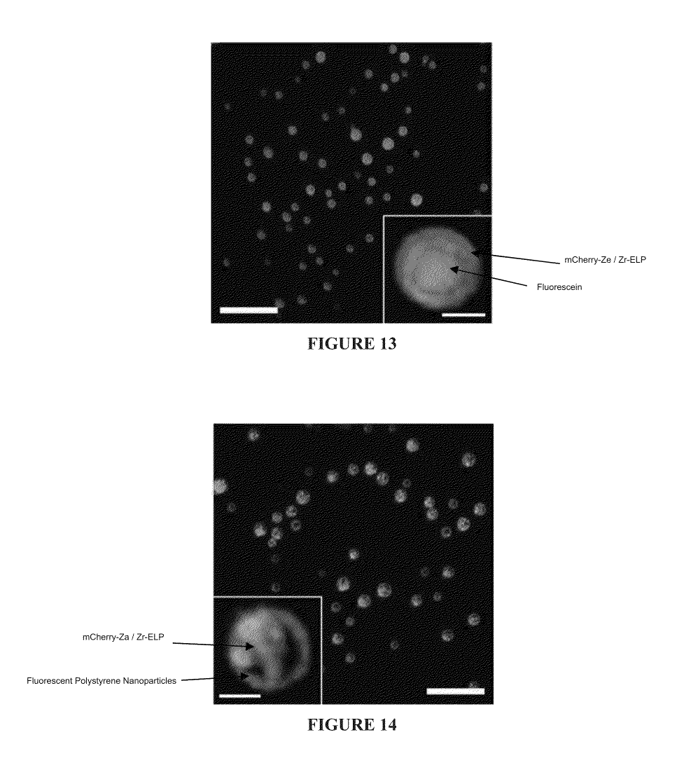

Encapsulation of small molecules was achieved by mixing with the protein amphiphiles, followed by inverse phase transition. Confocal microscopy showed the inner space of the resulting vesicles from mCherry-Z_{E} (1.5 μM) and Z_{R}-ELP (30 μM) with a salt concentration of 0.45 M was filled with fluorescein (FIG. 13). The level of fluorescein was maintained, which indicated low permeability through the vesicle membrane.

### Example 6

### Encapsulation of Nanoparticles by Protein Vesicles

### Materials and Methods

Materials

Fluorescent polystyrene nanoparticles (Fluoresbrite® YG Carboxylate Microspheres 0.50 μm) were purchased from Polysciences.

Methods

Encapsulation was performed following the same procedure as vesicle self-assembly, except that 125 μg/ml of fluorescent polystyrene nanoparticles (1.8×10⁹ particles/ml) were added to protein mixture solutions on ice before room temperature incubation.

### Results

When mCherry-ZE vesicles were assembled in the presence of carboxylated fluorescent polystyrene nanoparticles (diameter approximately 500 nm), the nanoparticles were observed to be located inside the resulting vesicles (FIG. 14). Confocal microscopy showed the green fluorescent particles were surrounded by the red fluorescent vesicular membrane. This result could be explained if the vesicle membrane provides a hydrophobic inner wall. Considering the low number density and length scale of the nanoparticles relative to vesicles, encapsulation should be driven by attractive interactions between the hydrophobic nanoparticles and inner wall of the membrane.

The protein vesicles can encapsulate cargo with multiple length scales: small molecules (˜10⁰ nm), proteins (˜10¹ nm), and nanoparticles (˜10² nm).

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A hollow, protein vesicle comprising:
a protein membrane surrounding a hollow core, wherein the protein membrane comprises a first and a second modular protein amphiphile, wherein the first modular protein amphiphile comprises a hydrophobic block and a first hydrophilic protein binding block and the second modular protein amphiphile comprises a variable polypeptide block and a second hydrophilic protein binding block that binds to the first hydrophilic protein binding block; and
wherein the first and second protein amphiphiles self-assemble to form the hollow, protein vesicle.

2. The hollow, protein vesicle of claim 1, wherein the hydrophobic block of the first modular protein amphiphile comprises elastin-like polypeptide.

3. The hollow, protein vesicle of claim 2, wherein the first and second hydrophilic protein binding blocks comprise a leucine zipper motif.

4. The hollow, protein vesicle of claim 3, wherein the first hydrophilic protein binding block comprises an arginine-rich leucine zipper motif and the second hydrophilic protein binding block comprises a glutamic acid-rich leucine zipper motif.

5. The hollow, protein vesicle of claim 1, wherein the second hydrophilic block comprises a catalytic domain of an enzyme, a targeting moiety, a fluorescent polypeptide, a Human Leukocyte Antigen polypeptide, a T cell receptor polypeptide, a detectable label, a globular polypeptide, an immunostimulatory polypeptide, or combinations thereof.

6. The hollow, protein vesicle of claim 5, wherein the enzyme is selected from the group consisting of a dehydrogenase, a reductase, a protease, a synthetase.

7. The hollow, protein vesicle of claim 5, wherein the targeting moiety is selected from the group consisting of an antibody, aptamer, ligand receptor, and fusion protein.

8. The hollow, protein vesicle of claim 7, wherein the antibody is selected from the group consisting of a monoclonal antibody, a polyclonal antibody, a diabody, a single chain antibody, and antigen binding fragments thereof.

9. A method for making hollow, protein vesicles comprising:
combining a plurality of first and second modular protein amphiphiles in a solution on ice;
adjusting the salt concentration to promote or allow inverse phase transition of the protein amphiphiles; and
incubating the salt-adjusted solution at room temperature to form hollow, protein vesicles.

10. The method of claim 9, wherein the first modular protein amphiphile comprises a hydrophobic block and a first hydrophilic protein binding block and the second modular protein amphiphile comprises a variable polypeptide block and a second hydrophilic protein binding block that binds to the first hydrophilic protein binding block.

11. The method of claim 10, wherein the hydrophobic block of the first modular protein amphiphile comprises elastin-like polypeptide.

12. The method of claim 11, wherein the first and second hydrophilic protein binding blocks comprise a leucine zipper motif.

13. The method of claim 12, wherein the first hydrophilic protein binding block comprises an arginine-rich leucine zipper motif and the second hydrophilic protein binding block comprises a glutamic acid-rich leucine zipper motif.

14. A hollow, protein vesicle produced according to the method of claim 9.

15. A loaded, protein vesicle comprising:
a protein membrane surrounding a core, wherein the protein membrane comprises a first and a second modular protein amphiphile, wherein the first modular protein amphiphile comprises a hydrophobic block and a first hydrophilic protein binding block and the second modular protein amphiphile comprises a variable polypeptide block and a second hydrophilic protein binding block that binds to the first hydrophilic protein binding block; and
wherein the first and second protein amphiphiles self-assemble around cargo to form the protein vesicle with the cargo loaded in the core of the protein vesicle.

16. The loaded, protein vesicle of claim 15, wherein the hydrophobic block of the first modular protein amphiphile comprises elastin-like polypeptide.

17. The loaded, protein vesicle of claim 16, wherein the first and second hydrophilic protein binding blocks comprise a leucine zipper motif.

18. The loaded, protein vesicle of claim 17, wherein the first hydrophilic protein binding block comprises an arginine-rich leucine zipper motif and the second hydrophilic protein binding block comprises a glutamic acid-rich leucine zipper motif.

19. The loaded, protein vesicle of claim 16, wherein the cargo comprises a drug, a protein, a contrast agent, nanoparticles, a fluorophore, a radiolabel, a therapeutic compound, an antioxidant, a growth factor, a cytokine, a chemoattractant, a nucleic acid or a combination thereof.

20. The loaded, protein vesicle of claim 19, wherein the drug is selected from the group consisting of a cytocidal compound, a chemotherapy agent, a cellular metabolism blocker, a cell cycle inhibitor, or a combination thereof.

21. The loaded, protein vesicle of claim 19, wherein the nucleic acid encodes a functional protein.

22. The nucleic acid of claim 19, wherein the nucleic acid is selected from the group consisting of double stranded DNA, RNA, siRNA, microRNA, single stranded DNA, and anti-sense DNA.


SEQUENCE LISTING (29 sequences)

SEQ ID NO: 1 - protein, 5 aa, Artificial Sequence
  Synthetic Peptide
VPGXG

SEQ ID NO: 2 - protein, 236 aa, Artificial Sequence
  mCherry Sequence
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 3 - protein, 238 aa, Artificial Sequence
  GFP Sequence
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 4 - DNA, 1150 bp, Artificial sequence
  Synthetic sequence
atgtctagagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggaggtagatttatgcatcctcttgtcatgagaagtcgaattgttcccattctgtgtgttgcagctacagatggagatacatagagatactcgtggattttgcttagtgttgagttttgttctggttgtgaactaaaagtttatacatttgcaggaaataaatagccttttgtttaaatcaaaaggtcttacctatgttagtgtgaagcattggatcccaaagaactccaaaatgcgatgaggcatatttaatcttgtctggactagtaacaggttgggatgaccacctgtgaagctccaacaggattgcctcctcacgcaatgtttgaggtctgatgttcaatagcttgttttgtttcactttgctttggactttcttttcgccaatgagctatgtttctgatggttttcactcttttggtgtgtagagaaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 5 - protein, 7 aa, Artificial sequence
  Synthetic or recombinant peptide
LXXXXXX

SEQ ID NO: 6 - protein, 21 aa, Artificial sequence
  Synthetic or recombinant peptide
LXXXXXXLXXXXXXLXXXXXX

SEQ ID NO: 7 - protein, 28 aa, Artificial sequence
  Synthetic or recombinant peptide
LXXXXXXLXXXXXXLXXXXXXLXXXXXX

SEQ ID NO: 8 - protein, 35 aa, Artificial sequence
  Synthetic or recombinant peptide
LXXXXXXLXXXXXXLXXXXXXLXXXXXXLXXXXXX

SEQ ID NO: 9 - protein, 43 aa, Artificial sequence
  Synthetic or recombinant peptide
LEIEAAALEQENTALETEVAELEQEVQRLENIVSQYRTRYGPL

SEQ ID NO: 10 - protein, 43 aa, Artificial sequence
  Synthetic or recombinant peptide
LEIRAAALRRRNTALRTRVAELRQRVQRLRNEVSQYETRYGPL

SEQ ID NO: 11 - protein, 25 aa, Artificial sequence
  SYNTHETIC PEPTIDE
VPGVGVPGVGVPGFGVPGVGVPGVG

SEQ ID NO: 12 - protein, 10 aa, Artificial sequence
  Synthetic or recombinant peptide
VLAVAVLAVA

SEQ ID NO: 13 - protein, 4 aa, Artificial sequence
  Synthetic or recombinant peptide
GSGS

SEQ ID NO: 14 - protein, 4 aa, Artificial sequence
  Synthetic or recombinant peptide
GGGS

SEQ ID NO: 15 - protein, 15 aa, ARTIFICIAL SEQUENCE
  SYNTHETIC OR RECOMBINANT PEPTIDE
GGGGSGGGGSGGGGS

SEQ ID NO: 16 - protein, 20 aa, ARTIFICIAL SEQUENCE
  SYNTHETIC OR RECOMBINANT PEPTIDE
GGGGSGGGGSGGGGSGGGGS

SEQ ID NO: 17 - protein, 14 aa, ARTIFICIAL SEQUENCE
  SYNTHETIC OR RECOMBINANT PEPTIDE
GGSGSGSGSGSGSG

SEQ ID NO: 18 - protein, 8 aa, ARTIFICIAL SEQUENCE
  SYNTHETIC OR RECOMBINANT PEPTIDE
DYKDDDDK

SEQ ID NO: 19 - protein, 6 aa, ARTIFICIAL SEQUENCE
  SYNTHETIC OR RECOMBINANT PEPTIDE
YPYDVP

SEQ ID NO: 20 - protein, 10 aa, ARTIFICIAL SEQUENCE
  SYNTHETIC OR RECOMBINANT PEPTIDE
ILKKATAYIL

SEQ ID NO: 21 - protein, 10 aa, ARTIFICIAL SEQUENCE
  SYNTHETIC OR RECOMBINANT PEPTIDE
EQKLISEEDL

SEQ ID NO: 22 - DNA, 39 bp, ARTIFICIAL SEQUENCE
  SYNTHETIC
tatcatagatctatggctagcaaaggagaagaactcttc

SEQ ID NO: 23 - DNA, 52 bp, ARTIFICIAL SEQUENCE
  SYNTHETIC
cagggatccactaccgcgaagattgtacagttcatccatgccatgtgtaatc

SEQ ID NO: 24 - DNA, 32 bp, ARTIFICIAL SEQUENCE
  SYNTHETIC
cttcgcggtagtggatccctggaaatcgaagc

SEQ ID NO: 25 - DNA, 32 bp, ARTIFICIAL SEQUENCE
  SYNTHETIC
tatcatagatctcagcggaccgtaacgggtac

SEQ ID NO: 26 - protein, 51 aa, ARTIFICIAL SEQUENCE
  SYNTHETIC OR RECOMBINANT PEPTIDE
LEIEAAALEQENTALETEVAELEQEVQRLENIVSQYRTRYGPLRSHHHHHH

SEQ ID NO: 27 - protein, 187 aa, ARTIFICIAL SEQUENCE
  SYNTHETIC
MKGSLEIRAAALRRRNTALRTRVAELRQRVQRLRNEVSQYETRYGPLGGGGSGGGGSGVPGVGVPGVGVPGFGVPGVGVPGVGVPGVGVPGVGVPGFGVPGVGVPGVGVPGVGVPGVGVPGFGVPGVGVPGVGVPGVGVPGVGVPGFGVPGVGVPGVGVPGVGVPGVGVPGFGVPGVGVPGVGVPGC

SEQ ID NO: 28 - protein, 301 aa, ARTIFICIAL SEQUENCE
  SYNTHETIC
MGGSRSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSKLRGSGSLEIEAAALEQENTALETEVAELEQEVQRLENIVSQYRTRYGPLRSHHHHHH

SEQ ID NO: 29 - protein, 302 aa, ARTIFICIAL SEQUENCE
  SYNTHETIC
MGGSRSMASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNLRGSGSLEIEAAALEQENTALETEVAELEQEVQRLENIVSQYRTRYGPLRSHHHHHH